(19) United States
(12) Patent Application Publication
(10) Pub. No.: US 2009/0074885 A1
(43) Pub. Date: Mar. 19, 2009

(54) Reversible Hydrophobic Modification of Drugs for Improved Delivery to Cells

(75) Inventors: Sean D. Monahan, Mazomanie, WI (US); Vladimir Subbotin, Madison, WI (US); Zane C. Neal, Cambria, WI (US); Vladimir G. Budker, Middleton, WI (US); Tatyana Budker, Middleton, WI (US)

Correspondence Address: ROCHE MADISON INC., 505 S. ROSA RD., MADISON, WI, 53719, US

(73) Assignee: Roche Madison Inc., Madison, WI (US)

(21) Appl. No.: 12/260,679

(22) Filed: Oct. 29, 2008

Related U.S. Application Data
(63) Continuation-in-part of application No. 10/929,697, filed on Aug. 30, 2004.
(60) Provisional application No. 60/985,700, filed on Nov. 6, 2007.
(60) Provisional application No. 60/501,189, filed on Sep. 8, 2003.
(60) Provisional application No. 60/520,426, filed on Nov. 14, 2003.
(60) Provisional application No. 60/513,707, filed on Oct. 23, 2003.
(60) Provisional application No. 60/558,753, filed on Apr. 1, 2004.

(51) Int. Cl. A61K 31/704; A61K 33/24; A61K 31/337; A61P 35/04; A61K 31/195

(52) U.S. Cl. 424/649; 514/564; 514/34; 514/449

(57) ABSTRACT

Described are drug formulations that increase regional delivery of the drugs to cells. Methods for reversibly increasing the hydrophobicity of a drug through hydrolytically labile attachment of a hydrophobic moiety and methods for delivering the modified drug to cells are described. Hydrophobic modification increases drug delivery, while lability minimizes entry of the drug into non-target cells.

### CROSS-REFERENCE TO RELATED APPLICATIONS

This application is a continuation-in-part of U.S. application Ser. No. 10/929,697, filed 30 Aug. 2004 and claims the benefit of U.S. Provisional Application 60/985,700 filed 6 Nov. 2007, application Ser. No. 10/929,697 claims the benefit of U.S. Provisional Application Nos. 60/501,189, filed Sep. 8, 2003, 60/520,426, filed Nov. 14, 2003, 60/513,707, filed Oct. 23, 2003, and 60/558,753, filed Apr. 1, 2004.

### BACKGROUND OF THE INVENTION

A variety of methods and routes of administration have been developed to deliver pharmaceuticals to their site of action. One of the general problems associated with drug delivery is balancing the ability to cross cell membranes with solubility in water. If a drug is too hydrophilic, it will be unable to cross the hydrophobic environment of the lipid cell membrane. If a drug is too lipophilic, it will aggregate or have limited solubility in an aqueous environment. A lipophilic drug could also be confined to the cell membrane if it does reach the cell. Most of the drug formulations are therefore amphiphilic, containing both hydrophilic and hydrophobic characteristics, or are formulated with the use of excipient(s) to aid in the delivery of the drug.

Although advances have been made in drug delivery, improvements are still needed in order to improve the therapeutic index of drugs. Cellular drug delivery by conventional water-soluble drug formulations is limited by three obstacles regardless of the route of administration: a) low partitioning through the cell lipid membrane, b) rapid clearance from a site of administration by the circulation, and c) redistribution throughout the body potentially leading to accumulation in unwanted tissue and systemic toxicity. Attempts to overcome the first two obstacles by increasing the dose of the drug increases systemic toxicity. Systemic toxicity is also a concern for drugs that exhibit higher levels of tumor/cellular uptake, and often limits the amount of drug that can be administered.

Recent efforts to improve the therapeutic index of drugs for tumor treatment have led to advancements in prodrug design and to the development of a variety of drug delivery systems. Delivery systems utilizing liposomal, polymeric conjugate, micelle, polymeric micelle, and nanoparticles have been described employing both active (receptor and antibody mediated) and passive (enhanced permeability and retention) tumor targeting. A particularly valuable component for the design of these advanced systems involves the use of hydrolytically or enzymatically labile chemical linkages in order to release the drug from the delivery system. Although a number of systems have been described, systems derived from cis-aconityl and hydrazone linkages have attracted the most interest.

Despite the advances in drug delivery, additionally approaches are necessary in order to effectively target drugs and improve uptake to cells of interest, most notably cancer cells. One approach that has generated interest is the use of regional or loco-regional drug treatment strategies. These strategies propose to achieve a high concentration of a drug at a target site by delivering the drug at or near the target site. Based on the rationale of first-pass drug extraction, a high drug concentration at the target site could increase the amount of drug uptake while at the same time decreasing normal systemic tissue exposure, therefore minimizing drug-related toxicities.

Several types of diseases, notably several types of cancers, have been treated with a regional treatment regiment. For example, the regional treatment of liver cancers has been explored. The liver is the predominant site for metastatic disease progression from a variety of tumor origins, including colorectal carcinoma, melanoma, and neuroblastoma, and is the primary site for hepatocellular carcinoma (HCC) and cholangiocarcinoma. Traditional systemic chemotherapy has demonstrated poor antitumor benefit and only marginal increases in survival. As a result, resection and transplantation remain the only curative options for patients with progressive liver disease. However due to disease recurrence, or vascular invasion and the presence of multifocal disease, these options might not be medically available.

As liver neoplasms grow, tumors reaching a diameter of 5-7 mm are predominantly perfused by a neovascularized hepatic arterial route. Normal liver parenchyma, however, is supplied mainly from the portal vein (75%). Exploitation of this difference motivated the development of loco-regional drug treatment strategies such as direct hepatic artery infusion (HAI). However, analysis of a multicenter randomized trial indicated no differences in overall survival between HAI and systemic chemotherapy administration, and recommended discontinued HAI utility outside the scope clinical trials. Another regional therapy has been developed consisting of transcatheter hepatic artery chemotherapy (TAC) via the femoral artery (bolus injection). In order to prolong drug contact with the tumor tissue, TAC has further been developed to include embolization (TACE). However, using conventional drugs, TACE has shown only modest patient benefit for both primary and secondary liver malignancies.

Additionally, a regional treatment for ovarian cancer has been investigated. Ovarian cancer is the second most common pelvic tumor and the leading cause of death from a gynecologic malignancy. Because of the lack of symptoms in the early stages, two thirds of patients present with advanced late-stage disease. Despite advances in surgical oncology, chemotherapy, and molecular biology, overall 5-year survival rates are still poor (approximately 30%).

Intraperitoneal chemotherapy (IPC) was introduced for peritoneal disseminated disease in an effort to direct high levels of chemotherapeutics to the peritoneal exposed tumor surface area. This treatment regime has been additionally modified as intraperitoneal perfusion chemotherapy (IPPC). IPPC removes unabsorbed drug from the peritoneal cavity in order to decrease systemic toxicity and allow for higher dose administration of the chemotherapeutics. However, only modest benefits in disease remission and patient survival have been achieved for either IPC or IPPC.

The limitations observed in both liver tumor and ovarian cancer chemotherapeutic treatments could be due, in part, to poor extraction of the drugs by the tumor tissue. Conventional chemotherapeutics generally exhibit low partitioning through lipid membranes, poor cellular uptake, and are cleared rapidly from the site of application, even when delivered locally in high concentration (as in the setting of HAI/TAC or IPC/IPPC therapy). Low extraction of drugs by the tumor tissue results in lack of anti-tumor benefit and a relatively high hepatic/systemic exposure and high toxicity profiles. Even for chemotherapeutics that exhibit higher levels of tumor/cellular uptake, concerns of systemic exposure often limit the amount of drug that can be administered. In either case, the limited patient benefit observed suggests that conventional local targeting is insufficient in eliminating tumor cells, and that poor first-pass drug extraction by the tumor tissue remains a serious issue.

The ability of chemotherapeutics to mediate cytotoxic activity is dependent on sufficient intracellular drug accumulation in the target cell. Intracellular drug levels are a function of the amount of drug are drug transported inside the cell (influx) and the amount of drug expelled from the cell (efflux). Drug uptake is determined by membrane transport, occurring through poorly defined mechanisms of passive diffusion and/or energy-dependent active transport. It has been proposed that approximately one-half of all drug uptake takes place by passive diffusion and the other half occurs by facilitated transport. It has been thought that lipid membranes represent a barrier for hydrophilic drug movement, but are not a barrier for hydrophobic drugs.

Hydrophobization or lipidization (modification of the therapeutic agent with hydrophobic moieties) has generated interest for both drug and peptide/protein delivery. Drug hydrophobization utilizing relatively stabile modifications such as esters and amides was shown to increase drug interactions with cellular membranes and has correlated with improved cellular uptake and lowered IC50 values. However, concerns remain involving both compound aggregation and embolization, and the sequestering of the drug in the cell membrane.

### SUMMARY OF THE INVENTION

Described are drug formulations that increase regional drug delivery to target cells. The drugs are modified with a hydrophobic group attached to the drug via a labile bond to make a prodrug. The resulting prodrug has increased hydrophobicity relative to the drug, and thus increased membrane binding and permeability. The resultant prodrug is stable in a suitable solvent, but is unstable in a suitable carrier solution. Just prior to administration of the prodrug to cells, the prodrug is mixed with a carrier solution. Rapidly reversible hydrophobization (RRH) increases cellular uptake of the prodrug in a first pass setting. Rapid reversibility or lability of the linkage protects other tissues by virtue of the loss of the membrane binding component.

In a preferred embodiment, we describe the transient hydrophobic conversion of a drug into a prodrug for delivery to cells via first-pass delivery. Hydrophobic conversion increases membrane permeability of the prodrug. Lability of attachment of a hydrophobic moiety to the drug provides for limited duration of this enhanced membrane permeability. Cleavage of the hydrophobic moiety after the association of the prodrug with the cell allows interaction of the unmodified drug with cellular components. Cleavage of the hydrophobic moiety on the prodrug outside the cell decreases the ability of the drug to enter cells and thus decreases undesired effects of the drug, such as toxicity, in non-target, i.e., non-first-pass, cells. A preferred hydrophobic moiety comprises a silazane. Another preferred hydrophobic moiety comprises a maleamic acid.

In a preferred embodiment we describe a method for delivering a hydrophobic drug or prodrug to a cell comprising: providing a prodrug that is soluble in an organic solvent, and injecting the prodrug in an organic solvent into a suitable mixing chamber designed to mix the organic solvent with a aqueous carrier solution just prior to delivery of a combined delivery solution to the cell. A suitable mixing chamber rapidly mixes the organic solvent with the aqueous carrier solution without producing laminar flow of the organic and aqueous solvents.

In another preferred embodiment we describe a method for delivering a hydrophobic drug or prodrug to a cell comprising: providing a prodrug that is stable in its dissolving solvent, and injecting the prodrug in its dissolving solvent into a suitable mixing chamber designed to mix the dissolving solvent with a aqueous carrier solution to form a combined delivery solution just prior to delivery of a combined delivery solution to the cell. The aqueous carrier solution is a solution in which the prodrug is not stable. A suitable mixing chamber rapidly mixes the prodrug dissolving solvent with the aqueous carrier solution without producing laminar flow of the dissolving solvent and aqueous solvents.

In a preferred embodiment, we describe compositions comprising: a prodrug that contains one or more hydrophobic groups attached to the drug via a labile bond, wherein the prodrug is soluble in an organic solvent. Hydrophobic modification increases delivery of the drug to a cell interior. Lability results in rapid regeneration of the unmodified drug. The hydrophobic prodrug can be delivered to a cell by mixing the prodrug, in an organic solvent, with a sufficient amount of an aqueous carrier solution just prior to administration of the prodrug to the cell, cell container, or tissue. A preferred hydrophobic prodrug comprises a hydrophobic silazane modified drug. Another preferred hydrophobic prodrug comprises a hydrophobic maleamic acid.

In a preferred embodiment, we describe a method for increasing delivery of a drug, such as an anti-tumor or anti-cancer drug, to tumor or cancer cells comprising: hydrophobically modifying the drug with one or more hydrophobic groups attached to the drug via a hydrolytically labile bond to make a prodrug, mixing an organic solution containing the prodrug with a carrier solution by injecting the solutions though a mixing chamber just prior to delivery, and administering the combined solutions at or near the tumor cell.

In another preferred embodiment, we describe a method for increasing delivery of a drug to tumor cells comprising: hydrophobically modifying the drug with one or more hydrophobic groups attached to the drug via a hydrolytically labile bond to make a prodrug, mixing the dissolving solvent containing the prodrug with a carrier solution by injecting the solutions though a mixing chamber just prior to delivery, and administering the combined solutions at or near the tumor cell.

In another preferred embodiment, we describe a method for the hydrophobic modification of a mixture of drugs (a drug library) with hydrophobic groups attached to the drugs via a hydrolytically labile bond to make a prodrug library, and the delivery of this prodrug library to cells. The hydrophobic prodrug library can be delivered to a cell by mixing the prodrug library, in an organic solvent, with a sufficient amount of an aqueous carrier solution just prior to administration of the prodrug library to the cell, cell container, or tissue.

In yet another preferred embodiment, we describe a method for the hydrophobic modification of a mixture of drugs (a drug library) with hydrophobic groups attached to the drugs via a hydrolytically labile bond to make a prodrug library, and the delivery of this prodrug library to cells. The hydrophobic prodrug library can be delivered to a cell by mixing the prodrug library, in a dissolving solvent, with a sufficient amount of an aqueous carrier solution just prior to administration of the prodrug library to the cell, cell container, or tissue.

In yet another preferred embodiment, we describe a method for the hydrophobic modification of a drug or mixture of drugs with a hydrophobic group attached to the drug via a labile bond to make a prodrug or mixture of prodrugs, wherein the labile bond is labile in response to a reaction by an agent, and delivery of this prodrug(s) to a cell. These hydrophobic prodrug(s) can be delivered to a cell by mixing the drug, in an organic solvent, with a sufficient amount of an aqueous carrier solution just prior to administration of the prodrug to the cell, cell container, or tissue. The agent can be a natural component of the cell or the environment of the cell or an agent added to the carrier solution.

In yet another preferred embodiment, we describe a method for the hydrophobic modification of a drug or mixture of drugs with a hydrophobic group attached to the drug via a labile bond to make a prodrug or mixture of prodrugs, wherein the labile bond is labile in response to a reaction by an agent, and delivery of this prodrug(s) to a cell. These hydrophobic prodrug(s) can be delivered to a cell by mixing the drug, in dissolving solvent, with a sufficient amount of an aqueous carrier solution just prior to administration of the prodrug to the cell, cell container, or tissue. The agent can be a natural component of the cell or the environment of the cell or an agent added to the carrier solution.

Further objects, features, and advantages of the invention will be apparent from the following detailed description when taken in conjunction with the accompanying drawings.

### BRIEF DESCRIPTION OF THE DRAWINGS

FIG. 1. Structures for PI (I), RRH-PI (BDMODS-PI (II), C12PMMA-PI (III)) prodrugs, stable PI derivative (C12CON-PI (IV)), and C12PMMA-melphalan (V).

FIG. 2. Illustrations of the chemical structures of: Cicplatin (CP), BDMODS-CP, Melphalan, BDMODS-Melphalan, the maleamic acid derivative CDMC12-Melphalan, Doxarubicin, DMODS-Dox, and the maleamic acid derivative CDMC12-Dox.

FIG. 3. Passive mixing chamber with colliding flows. The carrier solution (aqueous carrier solution and RRH-prodrug in solvent (drug carrier solvent) are delivered to the chamber by independent syringe pumps, with passive mixing from colliding flows. (A) Diagram, (B) Photo.

FIG. 4. Delivery of PI (I), BDMODS-PI (II), and C12CON-PI (IV) to various cell cultures. Drug/RRH-Prodrug (20 μl of 7.48 mM solution in DMSO) was mixed with ITG (200 μl), and added to the cells. After 30 sec, the drug solution was removed (aspirated) and 2 ml growth media was again added to the cells. The cells were immediately imaged with an Axiovert S100 fluorescent microscope (Zeiss) and the same fields were imaged with phase contrast illumination and in the rhodamine fluorescence channel using identical settings (Panels a-d). Alternatively, SK-OV-3 cells were immediately imaged (panel e) on an Axioplan2 fluorescent microscope (Zeiss), or returned to the incubator for 1 h prior to imaging (panels F). For imaging, the cover slip was removed and inverted on one drop of media. The same fields were imaged with phase contrast illumination and in the fluorescein fluorescence channels using identical settings. Panels a,b—Treatment of Hepa 1-6 cells with PI (a) or II (b) (magnification=32×). Panels c,d—Treatment of SK-OV-3 cells with PI (c) or II (d) (magnification=10×). Panels e,f—Treatment of SK-OV-3 cells with IV (E, imaged immediately following treatment) or IV (F, imaged 1 h following treatment), magnification=32×. Panel g—Flow Cytometry was conducted on Jurkat cells following treatment with PI (Control runs 1-4, no OS, no mixing chamber), and on a suspension of Jurkat cells in ITG, treated with PI (PI runs 1-4) or II (BDMODS-PI runs 1-4) through the mixing chamber. The results represent a histogram of the relative PI intensity of all single cell events. Panel h—Treatment of Hepa 1-6 cells with II then treated with Calcein AM to determine live cells (magnification=20×).

FIG. 5. (A) Images of SK-OV-3 cells treated with: 1a&b—unmodified propidium iodide (PI); 2a&b—BDMODS-PI; 3a&b—C12PMMA-PI; or 4a&b—pre-hydrolyzed BDMODS-PI. 1a-4-a: images under phase contrast illumination. 1b-4-b: images of the same fields under fluorescent illumination with rhodamine filter. (B) Images of Jurkat cells treated with (i) propidium iodide or (ii) C12PMMA-PI. Top panels show cells under phase contrast illumination. Bottom panels show the same field of cells under fluorescent illumination with rhodamine filter.

FIG. 6. Bar graph illustrating antiproliferative/cytotoxic effect of prodrugs on B16 murine melanoma cells as measured by CellTiter-Glo luminescent cell viability assay.

FIG. 7. Confocal images illustrating propidium iodide delivery to cells in vivo following treatment with BDMODS-PI. Targeting of cells exposed in peritoneal cavity in normal ICR mice. (A, B, C) intraperitoneal application RRH-PI to normal peritoneal organs. (A) Fallopian tube; (B) Jejunum; (C) Small monocyte infiltrate in visceral mesentery. (D) Application of unmodified-PI on jejunum. Upper left panels—fluorescence of DNA-intercalated PI, Upper right panels—actin stained with Phalloidin Alexa 488, Lower left panels—nuclear stain with ToPro-3, Lower right panels—composite images. Frozen sections, LSM 510 confocal microscopy, bar=100 microns.

FIG. 8. First-pass targeting of peritoneal disseminated ovarian cancer in mouse with RRH-PI. Peritoneal targeting was performed via peritoneal perfusion with aspiration. (A) Targeting of multiple cell layers in large ovarian tumor. (B) Targeting of tumor tissue growing on colon wall. (C) Targeting of mesenteric micrometastasis. (D) Targeting of tumor cell cluster growing on and invading large bowel. (E-F) Heart & lung tissues of animal that received RRH-PI via intraperitoneal perfusion. Upper left panels—fluorescence of DNA-intercalated PI, Upper right panels—actin stained with Phalloidin Alexa 488, Lower left panels—nuclear stain with ToPro-3, Lower right panels—composite images. Frozen sections, LSM 510 confocal microscopy, bar=100 microns.

FIG. 9. Pathological features of mouse model of disseminated peritoneal ovarian cancer, 5 wks after nude-Foxn1nu mice inoculation with human SK-OV-3 cancer cells. (A) Micro tumor growth on duodenal mesentery (×100). (B) Loose cell organization of mesentery tumor (×630). (C) Loose tumor cell growth on duodenal wall and pancreas. (D) Tumor cell growth on mesenteric lymph node. (E) Tumor cell growth on abdominal surfaces of liver. (F) Tumor cell growth on the diaphragm with invasion, all ×200. Paraffin sections, H&E stain.

FIG. 10. Confocal images following IPPC of C12PMMA-PI: (A) Surface of a large peritoneal tumor, and (B) a micro-ovarian tumor on the surface of the colon, ×630, 5 weeks post SK-OV-3 cell inoculation. Propidium iodide (upper left panels of A and B), ToPro-3 nuclear stain (lower left panels of A and B); Actin stained with Phalloidin Alexa 488 (upper right panel of A and B).

FIG. 11. Fluorescent images of liver sections following injection of modified propidium iodide (BDMODS-PI; A,B & D) or unmodified propidium iodide (C). (A-B)—Nuclei in MC38 metastases are strongly labeled with PI, as well as arteries and some adjunct cells following hepatic artery delivery. (C) Few MC38 metastases labeled in mice injected with unmodified PI. (D) No labeled cells in MC38 metastases following portal vein injection of BDMODS-PI. Images in the left column show propidium iodide fluorescence. Images in the right column show cell auto-fluorescence. Arrowheads in (C) and (D) indicate border of tumor. HV=hepatic vein. A=100×; B, C, D=200×.

FIG. 12. Delivery of BDMODS-PI to mouse liver with colon metastases. Left—×400 confocal image of liver with colon carcinoma tumors, arrow indicates portal tract with artery labeled, arrowheads indicate liver metastasis with vast majority of cells labeled. Right—×630 confocal image taken from the middle of metastasis, showing nearly all cells labeled with reported drug. Upper left panels—propidium iodide signal. Upper right panels—actin stained with Alexa 488. Lower left panels—ToPro-3 nuclear dye. Lower right panels—composite images.

FIG. 13. Days of survival following LABI delivery of C12PMMA-PI (III) or hydrolyzed C12PMMA-PI to C57BL mice. Following three weeks of MC38 tumor development C12PMMA-PI or Hydrolyzed C12PMMA-PI (0.150 μmol in 20 μl DMSO, 200 μl ITG) were delivered by LABI. The mouse abdomen was closed 4 min after drug treatment and the animals were monitored for survival time.

FIG. 14. First-pass delivery of labile hydrophobic drugs to: (A) hepatic artery endothelial and smooth muscle cells; (B) Gall bladder vascular and epithelial cells; (C) bile duct epithelia and nearby hepatocytes; (D) hepatocytes; (E) endothelial cells and neurons; (F) mouse liver containing metastises following injection of modified propidium iodide into the portal vein; (G) hepatic artery endothelia, smooth-muscle cells and tumors cells staining with modified propidium iodide; (H) ureter transitional epithelia; (I) renal pelvis transitional epithelia; (J) beginning renal pelvis epithelia; (K) collecting tubules; and, (L) cornea epithelia.

### DETAILED DESCRIPTION

We describe drug formulations and processes for delivering drugs into cells via a first-pass effect comprising: reversibly attaching one or more hydrophobic moieties to the drug via a very labile linkage to form a prodrug and bringing the prodrug into contact with the cells. The hydrophobic attachment imparts enhanced membrane association and permeability to the drug, thereby allowing the drug to enter a cell. The half-life of the hydrophobic attachment is comparable with the time necessary for first-pass delivery following single-bolus injection or the time necessary for drug diffusion after topical application. Thus, the prodrug is capable of this enhanced membrane association and permeability of a target cell for only a limited period of time. In one embodiment, the linkage attaching the hydrophobic group to the drug is stable in a compatible organic solvent but hydrolytically unstable in an aqueous environment. In another embodiment, the linkage attaching the hydrophobic group to the drug is more stable (longer half-life) in a basic environment but less stable as the pH is lowered. Because of the instability of the hydrophobic modification, prodrug that enters a cell rapidly reverts to the original drug molecule which is then free to interact with target molecules. Prodrug that does not interact with cell membranes during first-pass rapidly reverts to the less membrane permeable drug through loss of the hydrophobic moiety. Reversion limits delivery of the drug into non-targeted cells and tissues thus limiting systemic toxicity.

The described drug modifications and processes can be used to enhance cellular accumulation of a chemotherapeutic drug in tumor tissue and thereby decreasing the amount of the delivered dose that non-targeted cells are exposed to, thereby decreasing systemic toxicity. The chemotherapeutic, or anti-neoplastic, is transiently converted into a lipophilic or hydrophobic prodrug by attaching one or more hydrophobic moieties to the drug by labile bonds. Conversion of the drug to a prodrug promotes greater interaction with a cellular membrane. Rapid hydrolysis of the chemical linkage under physiological conditions restores the drug to the more membrane impermeable state associated with the parent drug. Transient lipophilic conversion facilitates enhanced drug uptake by tumor tissue and subsequent antitumor efficacy during first-pass delivery, while preserving low systemic toxicity by reversion to the parent drug prior to systemic exposure.

The hydrophobic modifications utilized in the prodrug formation are very labile, allowing for facile regeneration of the active drug within the cell. Because first-pass delivery serves to deliver more of the prodrug/drug to regional target cells, such as tumor cells, lowering of the overall dosing of the drug may be possible. The rapidly labile prodrugs, which are more cell permeable than the drug, rapidly revert to the less membrane permeable drug, thereby exposing non-target cells to the drug form rather than the more membrane permeable prodrug. The result is a transient increase the therapeutic index of conventional chemotherapeutics while maintaining low systemic toxicity.

While hydrophobic modification of chemotherapy drugs to increase cellular interactions has been described in the art, we now show that the use of very labile hydrophobic modifications enable unique treatment scenarios with increased regional cell uptake of a modified drug following a single bolus injection while minimizing systemic exposure of non-target tissue to the active drug. These modifications are also useful for topical treatment of target cells while limiting drug exposure to non-target cells. We present an approach to the design of new drug formulations using reversible hydrophobic modifications and specialized delivery techniques that are capable of targeting compounds to desired tissues or organs while limiting interactions with non-target cells. The described chemistries and delivery methods also allow formulation of prodrugs which are more hydrophobic, leading to better cell uptake and tumor penetration. A degree of hydrophobicity necessary to achieve cell delivery can be used without requiring that the prodrug remain water soluble.

The lipophilic character of the prodrug, and thus its level of membrane interaction, will depend on the number and hydrophobicity of groups attached. Sufficient hydrophobicity is added to the drug to increase delivery of the resultant prodrug to cells. Hydrophobic groups indicate in qualitative terms that the chemical moiety is water-avoiding. Typically, such chemical groups are not water soluble, and tend not to hydrogen bond. Hydrocarbons are hydrophobic groups. If the hydrophobic group comprises an alkyl chain, the length of an alkyl chain group will affect the hydrophobicity of the group. Hydrophobic groups compatible with the described invention may be selected from the group comprising: an alkyl chain of 4 to 30 carbon atoms, which may contain sites of unsaturation; an alkyl group containing an alkyl chain and alkyl rings (aromatic and/or non aromatic); and steroids. The linkages can also be designed such that they posses different lability rates in order to influence prodrug stability in vitro and in vivo.

Limited stability of the drug modification allows for a local high concentration of modified drug that is able to enter cells in a first-pass region. A too rapid half-life results in ineffective target cell uptake. Conversely, a half-life of the prodrug that is too long leads to increased delivery of drug to non-target cells and tissues, potentially leading to systemic toxicity. The lability of the described linkages is potentially controllable through the choice of the pharmaceutically acceptable carrier solution. For example, the pH of the carrier solution can be adjusted with the use of an appropriate buffer in order to control the half-life of the prodrug. For drugs which can be modified with multiple hydrophobic groups, attachment of additional groups can not only increase the hydrophobicity of the drug, but also effectively increase the time required for complete hydrolysis. Controlling the incubation time of the drug between initial mixing with the carrier solution and initial contact with cells can also be used to influence the amount of time the lipophilic prodrug is present with cells. The rate of hydrolysis of the prodrug may be retarded upon interaction with the cellular membranes. The kinetic lability required for optimal delivery can be controlled through temperature or composition of the pharmaceutically acceptable carrier solution, the volume of the injection, the concentration of the injected prodrug, and the total amount of prodrug delivered.

We demonstrate the hydrophobic modification of amine-containing drugs via two different chemical linkages. An amine-containing drug has a nitrogen atom in the molecule that is amenable to modification. The amine can be a primary, secondary, or tertiary amine, or another nitrogen derivative such as an aniline. Other reactive groups on the drug may also be utilized for rapidly reversible attachment of a hydrophobic group. The requirement is that the hydrophobation be rapidly reversible and that reversal, cleavage of the hydrophobic group or groups from the drug, yields an active drug.

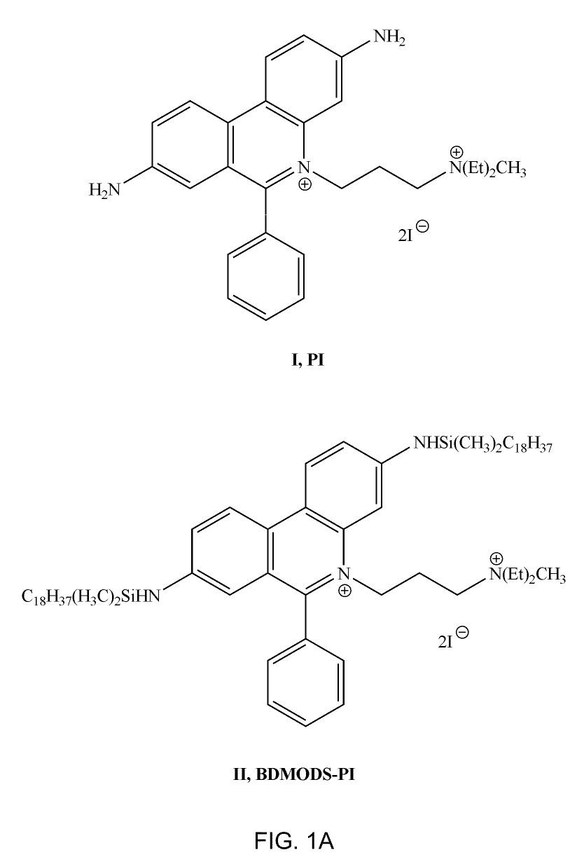

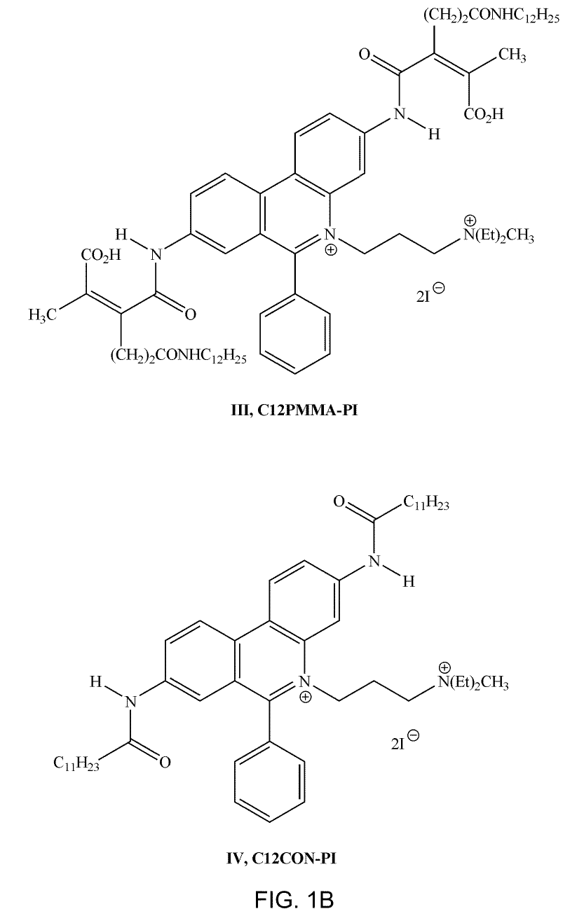

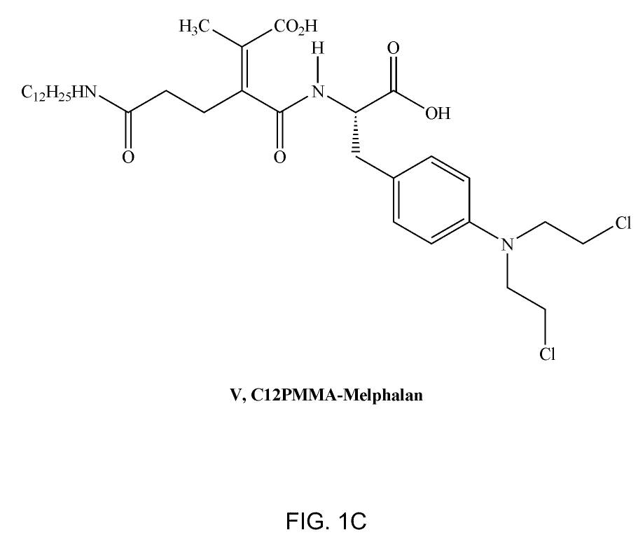

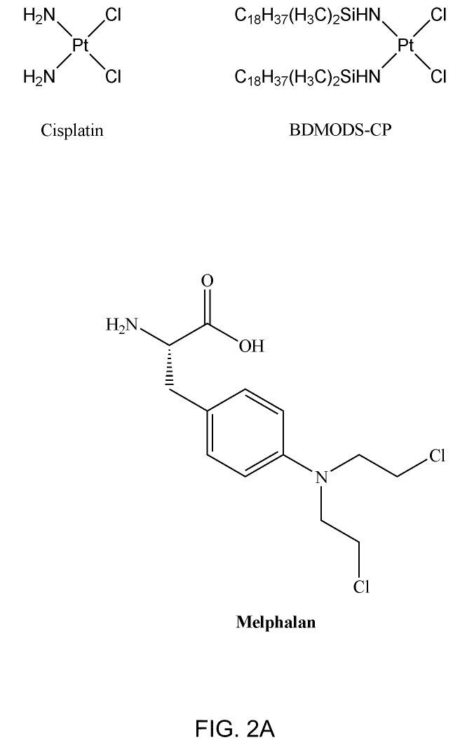

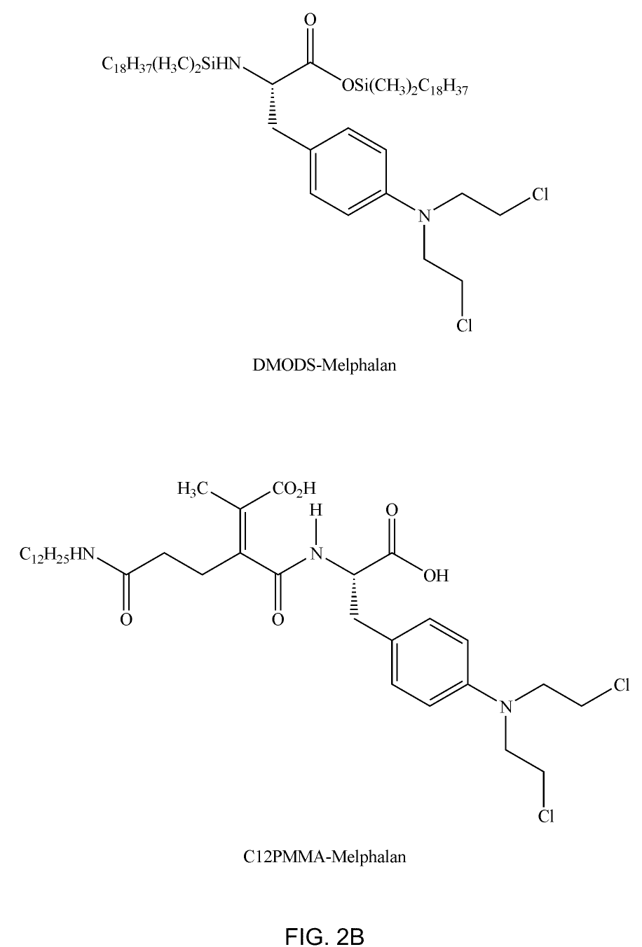

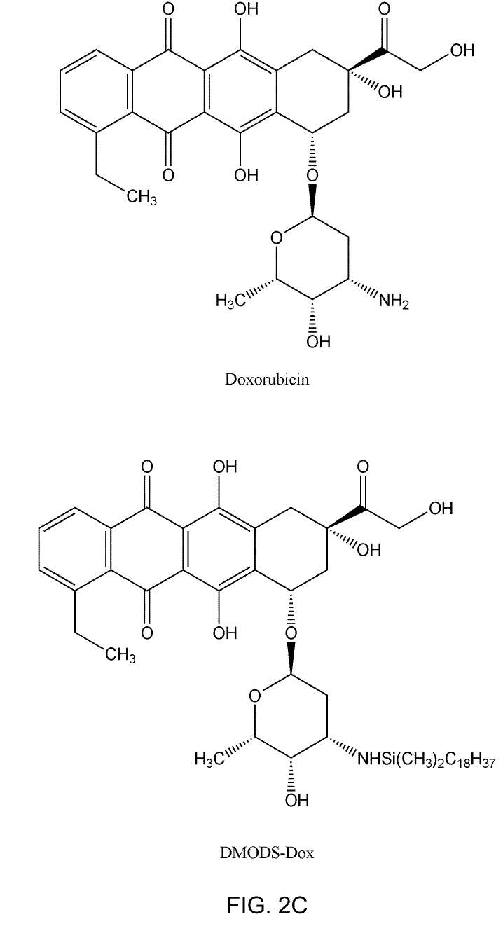

Amine containing drugs can be modified with silazanes. As an example, we show modification of drugs with chlorodimethyloctadecylsilane (DMODSiCl) to yield the corresponding dimethyloctadecylsilazane derivative as the hydrophobic prodrug (example shown in FIG. 1-2). The function of this group is to transiently attach hydrophobic groups to the drug molecule. The invention is meant to include other silazane derivatives. One skilled in the art will readily recognize that a variety of silazanes can be employed to impart transient hydrophobicity (for example, including but not limited to: trimethylsilyl and tert-butyl-dimethylsilyl groups).

The reaction between an amine and a chlorosilane is a well-known chemical modification which forms a silazane (or silylamine). Silazanes are known to hydrolyze rapidly in the presence of water to yield the original amine and a silanol or disilyl ether. Silazanes have generally been utilized in the field of ceramics or in organic synthesis as reagents for the silylation of other functional groups, most notably, the hydroxyl group. Because of its high lability, this modification has not found utility in biological applications. However, more stable heterosilanes have been employed as prodrugs. Examples include: a trimethylsilyl ether of testosterone; silabolin, a per-trimethylsilylated derivative of dopamine; carbosilane drugs; and silicon used as part of a delivery system. These examples employ a stable bond (carbon-silicon) or a slowly hydrolyzed bond (silicon-oxygen), not a rapidly hydrolyzed bond as found in the silazane. Silyl ethers have long been utilized as removable protecting groups in organic synthesis. The bond is hydrolytically labile under acidic conditions to yield an alcohol and a silanol or disilyl ether. Several factors control the hydrolysis rate of silyl ethers, for example the sterics of the silicon atom (i.e. the bulk of groups attached to silicon), and the pH of the solution. Silyzanes (with the exception of the known stable variants) hydrolyze much more readily than the corresponding oxygen variants (the silyl ethers).

For some drugs, direct silylation will prove inefficient. In such cases, other approaches can be used to form the prodrug. For example, in the case of cisplatin, DMODSiCl can be reacted with methylamine to form dimethyloctadecylsilyl-methylamine. This silazane can then be added to cisplatin or Pt(DMSO)₂-1,1-cyclobutanedicarboxylate to yield a labile cisplatin derivative. Silylation of a heterocyclic nitrogen atom is also possible.

Amine containing drugs can also be modified with maleic anhydrides possessing hydrophobic groups. As an example, we show modification of drugs with 2-(dodecyl)-propionamide-3-methylmaleic anhydride to yield the corresponding hydrophobic prodrugs. (example shown in FIG. 2) Maleic anhydrides have been previously utilized for reversible amine modification. The resulting maleamic acids are known to be stable under basic conditions, but hydrolyze rapidly under acidic conditions. For example, 2-propionic-3-methylmaleic anhydride (a carboxylic acid derivative) has been tested with glycinylalanine. The resulting maleamic acid has been shown to have a half-life of 2 min at pH 5 (k=0.3 min⁻¹). Given that aniline nitrogen's are generally less reactive than amines due to delocalization with the aromatic ring, it was expected that the lability of an aniline derived maleamic acid would be greater than that of the maleamic acid derived from a primary amine. As with the silazane, the purpose of the maleic anhydride is to transiently attach a hydrophobic groups to a drug molecule. One skilled in the art readily recognize that a variety of maleic anhydrides can be employed to impart transient hydrophobicity.

A great number of labile bonds are known to those skilled in the art, that could be utilized to attach a hydrophobic group or moiety to a drug molecule. The invention is also meant to encompass the use of hydrophobic drug modifications with these other types of hydrolytically labile bonds, when the derived prodrugs are then delivered via the delivery methods described in the present invention. Examples of additional labile bonds that may be used to attach the hydrophobic moiety to the drug include, but are not limited to: imines, ortho esters, acetals, aminals, silyl esters, and phosphosilyl esters.

A reversible or labile bond is a covalent bond other than a covalent bond to a hydrogen atom that is capable of being selectively broken or cleaved under conditions that will not break or cleave other covalent bonds in the same molecule. More specifically, a reversible or labile bond is a covalent bond that is less stable (thermodynamically) or more rapidly broken (kinetically) under appropriate conditions than other non-labile covalent bonds in the same molecule. Cleavage of the labile bond results in the formation of two molecules. For those skilled in the art, cleavage or lability of a bond is generally discussed in terms of half-life (t_{1/2}) of bond cleavage, or the time required for half of the bonds to cleave. Orthogonal bonds are bonds that cleave under conditions that cleave one and not the other. Two bonds are considered orthogonal if their half-lives of cleavage in a defined environment are 10-fold or more different from one another. Thus, reversible or labile bonds encompass bonds that can be selectively cleaved more rapidly than other bonds a molecule. Preferably, the invention encompasses hydrophobically modified drug formulations in which the half-life of the modification is less than or equal to 5 min in the delivery or carrier solution. Hydrophobic drug modifications with shorter half-lives in the delivery or carrier solution, less that 2 min, less than 1 min, less than 30 sec, or less than 20 sec, may be preferred. Lability is preferably selected to correspond to the time necessary to deliver the modified drug in a first pass setting.

The presence of electron donating or withdrawing groups can be located in a molecule sufficiently near the cleavable bond such that the electronic effects of the electron donating or withdrawing groups influence the rate of bond cleavage. Electron withdrawing groups (EWG) are atoms or parts of molecules that withdraw electron density from another atom, bond, or part of the molecule wherein there is a decrease in electron density to the bond of interest (donor). Electron donating groups (EDG) are atoms or parts of molecules that donate electrons to another atom, bond, or part of the molecule wherein there is an increased electron density to the bond of interest (acceptor). The electron withdrawing/donating groups need to be in close enough proximity to effect influence, which is typically within about 3 bonds of the bond being broken.

Another strategy for increasing the rate of bond cleavage is to incorporate functional groups into the same molecule as the labile bond. The proximity of functional groups to one another within a molecule can be such that intramolecular reaction is favored relative to an intermolecular reaction. The proximity of functional groups to one another within the molecule can in effect result in locally higher concentrations of the functional groups. In general, intramolecular reactions are much more rapid than intermolecular reactions. Reactive groups separated by 5 and 6 atoms can form particularly labile bonds due to the formation of 5 and 6-member ring transition states. Examples include having carboxylic acid derivatives (acids, esters, amides) and alcohols, thiols, carboxylic acids or amines in the same molecule reacting together to make esters, carboxylic and carbonate esters, phosphate esters, thiol esters, acid anhydrides or amides. Steric interactions can also change the cleavage rate for a bond.

Appropriate conditions are determined by the type of labile bond and are well known in organic chemistry. A labile bond can be sensitive to pH, oxidative or reductive conditions or agents, temperature, salt concentration, the presence of an enzyme, or the presence of an added agent. For example, increased or decreased pH may be the appropriate conditions for a pH-labile bond. For rapidly reversible pH-labile bonds, the pH of the carrier solution can be adjusted in order to effect the half-life of the prodrug formulation. In another example, oxidative conditions may be the appropriated conditions for an oxidatively labile bond. In yet another example, reductive conditions may be the appropriate conditions for a reductively labile bond.

The rate at which a labile group will undergo transformation can be controlled by altering the chemical constituents of the molecule containing the labile group. For example, addition of particular chemical moieties (e.g., electron acceptors or donors) near the labile group can affect the particular conditions (e.g., pH) under which chemical transformation will occur.

A labile linkage is a chemical compound that contains a labile bond and provides a link or spacer between two other groups. The groups that are linked may be chosen from compounds such as biologically active compounds, membrane active compounds, compounds that inhibit membrane activity, functional reactive groups, monomers, and cell targeting signals. The spacer group may contain chemical moieties chosen from a group that includes alkanes, alkenes, esters, ethers, glycerol, amide, saccharides, polysaccharides, and heteroatoms such as oxygen, sulfur, or nitrogen. The spacer may be electronically neutral, may bear a positive or negative charge, or may bear both positive and negative charges with an overall charge of neutral, positive or negative.

The methods described herein are also compatible with perfusion technology. In this context, perfusion refers to the deliberate introduction of fluid into a tissue. The fluid can be introduced into a vessel, tissue lumen, body cavity, such as the peritoneal cavity or in vitro cell container. More specifically, in isolated perfusion, the perfused tissue is isolated such that the introduced fluid does not reach non-target tissues. The isolated tissue can be flushed both before and after the perfusion to remove bodily fluid or introduced fluid from the tissue or region. Perfusion has been used to deliver anti-cancer agents into the blood vessels and tissues of an organ (liver or lung) or region of the body (usually an arm or a leg) using circulating bypass machines. Such a procedure is performed to treat cancer that has spread but is limited to an organ or region of the body. In the context of the present invention, the prodrug (dissolved in drug dissolving solvent) is mixed with an aqueous carrier solution in a mixing chamber and delivered to a the tissue to be perfused. An outflow line permits the prodrug delivery solution to perfuse through the cavity and exit through the outflow line. Because the prodrug and drug (resulting from loss of the hydrophobic group(s)) are removed from the tissue, it is possible to utilize prodrugs with a longer half-lives than in cases where the material is not removed following delivery. When the prodrug—drug is not removed, it is preferred to have a prodrug with a shorter half-life in order to protect downstream cells from the highly cell permeable prodrug. In the case of isolated perfusion, the prodrug is removed from the area of interest, thereby protecting cells outside the target region.

Rapidly reversible prodrugs may by synthesized in organic or other appropriate solvents. The described prodrugs are stable in the solvents but unstable in a carrier or delivery solution, such as an aqueous solution (for hydrolytically labile bonds). The reaction to form the modified drug can be conducted in a variety of solvents, however, a pharmaceutically acceptable injectable solvent is preferred. Furthermore, a solvent in which the modified drug can be purified from other components of the modification reaction (for example, hydrolyzed hydrophobic group, drying agents, and bases) is preferable to facilitate purification of the prodrug.

A variety of drugs can be modified according to the invention. In one embodiment, the drug is modified through an amine group on the drug. These drugs may be selected from the list comprising: chemotherapeutics, anti-neoplastic, doxorubicine (adriamycin), cisplatin (cis-diamminedichloroplatinum(II)), melphalan, and the tubulin polymerization agent paclitaxel. Additional functional groups that can be modified include alcohols, thiols, phosphates, and carboxylates. An active derivative of the parent drug, which contains a functional group suitable for modification may also be used. Examples of modified drugs include: cisplatin derivatives containing a heterocyclic nitrogen, anthracycline derivatives of doxorubicin, and amino or furanosyl substituted 5-fluorouracil.

We further describe methods for delivering labile prodrugs comprising: co-injecting the prodrug in an organic or other suitable solvent (a drug carrier solvent) together with a aqueous pharmaceutically acceptable carrier solution, though a mixing chamber. In one embodiment, the prodrug described herein comprise hydrophobic groups attached by very hydrolytically reactive linkages that requires synthesis and storage in organic solvents. However, toxicity concerns prohibit the direct delivery of drugs to cells in undiluted organic solvents. Therefore, mixing the organic solvents with a pharmaceutically acceptable aqueous carrier solution just prior to delivery by co-injection though a mixing chamber is performed.

The critical components of a suitable mixing chamber include: means by which to accurately deliver predetermined volumes of drug carrier solvent and aqueous carrier solution, means to rapidly and intimately mix the drug carrier solvent and aqueous carrier solution, and a means of delivering the combined liquid (delivery solution) to cells. Some commercial mixing chambers can result in laminar flows, without effective mixing of the drug carrier solvent with the carrier solution. If the drug carrier solvent is an organic solvent, incomplete mixing results in exposure of some cells to higher concentration of organic solvents that can lead to membrane damage. If the mixing is too slow, then the prodrug may be cleaved prior to contact with the cells. Any mixing chamber that provides adequate and rapid mixing of the drug carrier solvent with the aqueous carrier solution is suitable for use with the present invention.

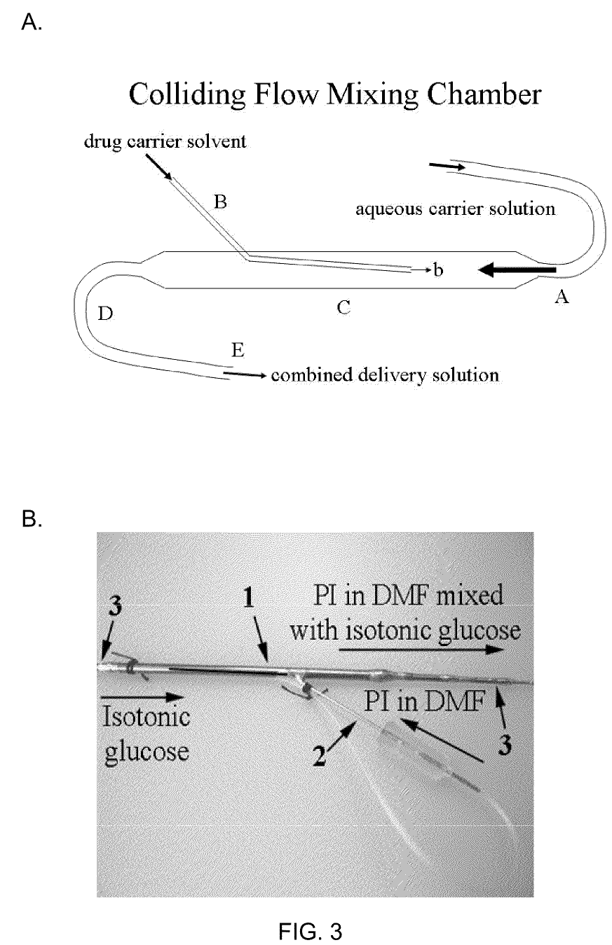

An example of a suitable mixing chamber is the colliding flow mixing microchamber shown in FIG. 3. The aqueous carrier solution and the drug carrier solvent are injected into a mixing chamber (C) though conduits (A) and (B) respectively. The direction of flow (b) of the drug carrier solvent into chamber (C) is in the opposite direction of the flow of the aqueous carrier solution into chamber (C), facilitating mixing of the two liquids. The combined delivery solution is then delivered to cells through vessel conduit (D) and instillation port (E). The volume of drug carrier solvent is generally much less than the volume of carrier solution. In one version of the mixing chamber, a Harvard Pump PHD 2000 with a 100 μl Hamilton syringe and a Harvard Pump PHD 2000 with a 1 ml Becton Dickinson syringe were used to accurately deliver small volumes to the chamber. Conduits (A), (B), and (D) may be rigid or flexible and may be made of any material than is suitable to convey the respective solutions and drugs. The length of conduit (D) may be varied in length to alter the amount of time the prodrug is in the aqueous carrier solution prior to delivery to the cells, thus modulating the half-life of the prodrug in the presence of the cells. A longer deliver route increases the incubation time and therefore decreases the half-life of the lipophilic modified drug in contact with the animal and/or on the cells. Suitable instillation ports (E) may be selected from the list comprising syringe needles and catheters.

In one embodiment, delivery solutions comprise about 1/10^{th} volume prodrug in solvent mixed with about 1 volume aqueous carrier solution (such as, but not limited to, Ringer's or isotonic glucose (ITG)). The total volume of prodrug-containing solvent to be delivered should be less than that which would cause toxicity from the solvent. The volume of carrier solution should be chosen to provide adequate total volume for the target area and provide adequate dilution of the prodrug-containing solvent. For larger animals, target areas, or cell containers, increased total volume is appropriate.

The membrane permeability and lability of the prodrug (i.e. the half-life of the modified drug) can be measured by monitoring the uptake of the prodrug by liposomes. The eluent from a suitable mixing chamber can be delivered to a solution containing liposomes whose composition approximates the plasma membrane of the target cells. The liposomes are then purified and the level of drug in the liposomes is measured. For DNA intercalating drugs, the liposomes can contain DNA to facilitate determination of drug uptake. In this manner, the acceptable volumes of solvent and carrier solution, as well as effectiveness of the mixing chamber can be analyzed.

The described processes and prodrugs are readily compatible with known techniques such as regional hepatic artery infusion (HAI) therapy, intraperitoneal chemotherapy (IPC), intraperitoneal perfusion chemotherapy (IPPC), transcatheter hepatic artery chemotherapy (TAC), transcatheter hepatic artery chemotherapy with embolization (TACE), and isolated organ or tissue perfusion. In isolated perfusion applications, potential systemic toxicity of the drug is further reduced because unabsorbed hydrolyzed drug is flushed from the isolated tissue (such as a peritoneal cavity) prior to restoration of normal fluid flow through the tissue. The describe processes and prodrugs are also compatible with topical delivery of the drug.

While the process may be described as a single bolus delivery of the prodrug, the process is not limited to a single administration. The process may be repeated to provide for increased levels of drug delivery. The term single bolus delivery is meant to be descriptive of first-pass delivery of the drug following an injection/application of a predetermined quantity of the prodrug.

The described prodrugs and methods can be used to generate an antitumor response against a variety of tumors, both primary and secondary, including, but not limited to, hepatocellular carcinoma, colon carcinoma, melanoma, ovarian carcinoma, and neuroblastoma. The utility of single bolus delivery is dependent on the ability of the drug agent to be preferentially exposed to the neoplastic tissue and penetrate the tumor cell membrane during first-pass delivery. Modification of anticancer drugs through labile attachment of hydrophilic moieties transforms relatively membrane impermeable drugs into lipophilic prodrugs that facilitate increased intracellular drug concentrations and enhanced anticancer responses.

For some cancers, such as peritoneal cancer, cancer cells and microtumors are invariably present together with the detectable and operable metastases. Their presence and continuous defoliation from primary and secondary malignancies represent one of the main impediments to the successful treatment of cancers such as peritoneal disseminated ovarian cancer. The disclosed prodrug formulations target all exposed cells, single cells, microinfiltrates, microtumors, and surface cells of larger peritoneal tumors, tumor cells suspended in peritoneal cavity or attached to or invading an organ or tissue. The described prodrugs also exhibit increased penetration of the drug into tumors compared to conventional drugs. We have observed drug penetration up to 500 μm (about 25 cell layers) within seconds. Thus, the described formulations provide for improved delivery of anticancer drugs to cancer cells in a variety of states. The described invention could therefore be utilized following cytoreductive surgery in efforts to slow or minimize reappearance of tumors.

Various types of tissues can be targeted using the described invention, depending on the type of delivery method utilized. For example, hepatocytes are targeted with a single bolus injection to the portal vein (occluded blood flow). Following a single bolus injection into the hepatic artery of normal mouse liver, targeting was evident in the hepatic artery endothelial and smooth-muscle cells, and in a few neighboring hepatocytes and sinusoidal cells. All biliary and gall bladder arteries, as well as bladder epithelium also were targeted. Bile duct cells together with some hepatocytes are targeted following a single bolus injection to the bile duct. Urinary tract cells are targeted (ureter transitional epithelium nuclei, renal pelvis transitional epithelium nuclei, including beginning renal pelvis, that is the source for transitional cell carcinoma, and a majority of collecting tubules, and other epithelial compartments) following a single bolus injection to the ureter. A single bolus injection into the carotid artery of a normal mouse resulted in the targeting of brain endothelial cells and both neurons and glial cells. Topical administration of prodrug results in delivery to the cells to which the prodrug is directly applied. For example, topical application to the cornea or to a skin or into the lumen of the intestine results in drug delivery to the cornea epithelium, or epidermis, or enterocytes respectively.

Given the unique vascular architecture of the liver, with portal blood supplying most normal hepatic tissue and hepatic arterial blood supplying tumors within the liver, the loco-regional delivery of RRH-therapeutics is uniquely suited to the treatment of primary and metastatic liver neoplasms.

To further increase delivery of drugs to cells, the described formulations and process may be combined with co-delivery of compounds known to modulate drug efflux pump efficiency. This co-delivery serves to increase drug retention in the cell.

The present invention is also applicable to the modification and delivery to cells of mixtures of drugs, also know as drug libraries. Most drugs contain nitrogen or oxygen atoms within the molecule that aid in the solubility of the drug in aqueous solutions. These atoms can be hydrophobized according to the procedures outlined in this specification. The drug library can be taken up in an appropriate organic solvent such as DMF or DMSO, and be subjected to hydrophobic modification such as outlined for a single compound. The derived prodrug library can then be applied to cells as outlined for a single prodrug.

The present invention is also applicable to a method for the hydrophobic modification of a drug or mixture of drugs via the attachment of a labile hydrophobic group to the drug wherein the hydrophobic group is labile in response to a reaction of an agent. For example the hydrophobic group can contain a disulfide bond which, upon entry to the cell, will be cleavable by the cellular agent glutathione. Hydrophobic groups compatible with the described invention contain a disulfide bond at or within 4 carbon atoms of the point of attachment of the group to the drug molecule that is susceptible to reduction by glutathione. The disulfide system also possesses a hydrophobic group on one side of the disulfide bond that may be selected from the group comprising: an alkyl chain of 4 to 30 carbon atoms, and can contain sites of unsaturation, an alkyl group containing an alkyl chain and alkyl rings (aromatic and/or non aromatic), and steroids. The linkages can also be designed such that they posses different lability rates in order to influence prodrug stability in vitro and in vivo.

The term drug in the present invention is also meant to include the pharmaceutically acceptable salt of the drug. Pharmaceutically acceptable salt means both acid and base addition salts. A pharmaceutically acceptable acid addition salt is a salt that retains the biological effectiveness and properties of the free base, is not biologically or otherwise undesirable, and is formed with inorganic acids such as hydrochloric acid, hydrobromic acid, sulfuric acid, nitric acid, phosphoric acid and the like, and organic acids such as acetic acid, propionic acid, pyruvic acid, maleic acid, malonic acid, succinic acid, fumaric acid, tartaric acid, citric acid, benzoic acid, mandelic acid, methanesulfonic acid, ethanesulfonic acid, p-toluenesulfonic acid, salicylic acid, trifluoroacetic acid, and the like. A pharmaceutically acceptable base addition salt is a salts that retains the biological effectiveness and properties of the free acid, is not biologically or otherwise undesirable, and is prepared from the addition of an inorganic organic base to the free acid. Salts derived from inorganic bases include, but are not limited to, sodium, potassium, calcium, lithium, ammonium, magnesium, zinc, and aluminum salts and the like. Salts derived from organic bases include, but are not limited to, salts of primary secondary, and tertiary amines, such as methylamine, triethylamine, and the like.

### EXAMPLES

### General

All chemical reactions were carried out under a nitrogen or argon atmosphere using flame dried glassware. Anhydrous N,N-dimethylformamide (DMF, Aldrich) and anhydrous dimethyl sulfoxide (DMSO, Aldrich) were used without further purification. Potassium carbonate (Aldrich) and molecular sieves (3 Å, Aldrich) were flame dried under vacuum prior to use. Propidium iodide (PI, 95%, Aldrich) was used without further purification. 1H NMR spectroscopy was performed on a Bruker AC+ 250, a Bruker AC+ 300, or a Varian Unity INOVA 400 spectrometer. Mass analysis was conducted on a PE Sciex API 150EX mass spectrometer. Cells were purchased from ATCC (Manassas, Va.), unless otherwise noted, and cultured according to the distributor's instructions.

### Example 1

Labile (rapidly reversible) and non-labile hydrophobic modifications of propidium iodide. Propidium iodide was utilized as a model reporter-drug. This membrane impermeable reporter drug is routinely used as a fluorescent agent to visually identify cells possessing compromised membranes. Cells with intact cellular membranes effectively exclude propidium iodide. Propidium iodide exhibits a 20-30-fold enhanced fluorescence upon intercalation into DNA, facilitating detection of propidium iodide positive (PI⁺) cells. The ability to deliver a fluorescent test drug to tumors provides a valuable visual tool in evaluating many experimental parameters. PI possesses two amino groups at the 3 and 8 positions of the phenanthridinium ring system that are available for modification

A. Synthesis of rapidly reversible 3,8-bis[((dimethyl)octadecylsilazyl) amine] 5-(3,3-diethyl-3-methylammonium propyl) 6-phenyl phenanthridinium diiodide (BDMODS-PI, II, FIG. 1). To propidium iodide (I) was added solid chloro(dimethyl)octadecyl-silane (6 eq, 95%, Aldrich), K₂CO₃ (10 eq), and molecular sieves (3 Å, 10 wt eq). To the resulting mixture was added anhydrous DMF or DMSO (7.5-15 μmol/ml final concentration), and the resulting suspension was heated at 60° C. for 12 h (hour). The suspension was cooled to ambient temperature, centrifuged, filtered (0.2 μm Nylon membrane) under inert atmosphere, and precipitated in diethyl ether to afford II (Propidium iodide, bis-(dimethyl)octadecyl silazane, BDMODS-PI) as a purple solid. 300 MHz NMR (N,N-dimethylformamide-d7, 99.5%, ppm) δ 8.72 (2H, dd, J=9.2, 6.6) 8.38 (1H, d, J=1.7) 7.93-7.80 (5H, m) 7.74 (1H, dd, J=9.1, 2.3) 7.48 (1H, dd, J=9.1, 1.7) 6.66 (1H, s) 6.46 (1H, d, J=2.3) 6.13 (1H, s) 4.59 (2H, m) 4.66-4.55 (2H, m) 3.88-3.75 (2H, m) 3.58-3.40 (2H, q, J=7.2) 3.21 (3H, s) 2.62-2.45 (2H, m) 1.40-1.18 (70H, m) 0.90-0.84 (6H, m) 0.55-0.49 (4H, m) 0.3 (12H, s). Mass analysis of II indicates the mass for PI (and fragmentation products), presumably due to the hydrolytic loss of the silyl group upon injection into the instrument. In DMSO λₑₓ max=460, λₑₘ max=638.

In some preparations, 1H NMR analysis (300 MHz, N,N-dimethylformamide-d7) indicated the formation two additional compounds as minor components in the reaction mixture (<10%). A trisilylated PI was observed (based on integration), arising from two silylation reactions taking place on one of the PI amino groups. The second minor component was identified as the phenanthridinium salt of BDMODS-PI (verified by independent synthesis, from the reaction of PI and chloro(dimethyl)octadecylsilane in dichloromethane in the absence of a base). These compounds would be expected to show similar cell internalization and hydrolysis properties as II, and were therefore not thought to be significant contaminants.

B. Synthesis of rapidly reversible 3,8-bis-[2-(2 carboxyethylidene)-4-(dodecarbamoyl)-1-oxo-butylamine] 5-(3,3-diethyl-3-methylammonium propyl) 6-phenyl phenanthridinium diiodide (C12PMMA-PI, III, (also 2-(dodecyl)-propion-amide-3-methylmaleic anhydride, CDMC12-PI, as named in U.S. application Ser. No. 10/929,697), FIG. 1). A second modification utilizes an amidation reaction between PI and the disubstituted maleic anhydride derivative N-dodecyl-3-(4-methyl-2,5-dioxo-2,5-dihydro-furan-3-yl)-propionamide (C12PMMA), to form the bis-maleamic acid III. Maleamic acids are known to be labile under acidic pH, reverting to the amine and the cyclic anhydride, with derivatives of disubstituted maleic anhydride showing the most rapid rate of hydrolysis (t_{1/2}˜5 min at pH 5)
i) Synthesis of N-Dodecyl-3-(4-methyl-2,5-dioxo-2,5-dihydro-furan-3-yl)-propionamide (C12PMMA). To a solution of 3-(4-methyl-2,5-dioxo-2,5-dihydro-furan-3-yl)-propionic acid (800 mg, 4.34 mmol) in dichloromethane (22 ml), was added oxalyl chloride (0.41 ml, 4.56 mmol, Aldrich) dropwise under nitrogen, and the resulting solution was stirred at ambient temperature. After 6 h, the solution was concentrated under reduced pressure. The resulting oil was suspended in dichloromethane (22 ml) and dodecyl amine (1.10 ml, 4.78 mmol, Aldrich) was added followed by diisopropylethylamine (0.83 ml, 4.78 mmol, Aldrich). After 4 h, the solution was concentrated under reduced pressure. The resulting reside was taken up in EtOAc (150 ml) and washed 2×HCl (20 ml, 1N), 1×H₂O, dried (Na₂SO₄), filtered and concentrated to afford a white solid. The solid was purified by flash chromatography on silica gel (40×160 mm, EtOAc/Hexanes 1:1 eluent) to afford 1.04 g C12PMMA (68%) as a white solid. 400 MHz 1H NMR (CDCl₃, 99.8%, ppm) δ 5.720 (1H, s) 3.20 (2H, dt, J=6.4, 7.0) 2.79 (2H, t, J=7.0) 2.530 (2H, t, J=7.0) 2.13 (3H, s) 1.55-1.42 (2H, m) 1.34-1.24 (18H, m) 0.88 (3H, t J=7.0). 13C NMR (CDCl₃, 99.8%, ppm) δ170.509, 166.194, 166.156, 143.007, 142.465, 39.974, 33.093, 32.116, 29.832, 29.786, 29.756, 29.733, 29.549, 29.481, 27.106, 22.890, 20.645, 14.321, 9.876. Molecular ion+1 calculated for C₂₀H₃₃NO₄=352.2, found m/e=352.2.
ii). Synthesis of 3,8-bis-[2-(2 carboxyethylidene)-4-(dodecarbamoyl)-1-oxo-butylamine] 5-(3,3-diethyl-3-methylammonium propyl) 6-phenyl phenanthridinium diiodide (III, C12PMMA-PI). To propidium iodide was added solid C12PMMA (3 eq), K₂CO₃ (10 eq), and molecular sieves (3 Å, 10 wt eq). To the resulting mixture was added anhydrous DMF or DMSO (7.5-15 μmol/ml final concentration), and the resulting suspension was heated at 60° C. for 12 h. The suspension was cooled to ambient temperature, centrifuged, filtered (0.2 μm Nylon membrane) under inert atmosphere, and precipitated in diethyl ether to afford III (propidium iodide bis-(2-(dodecylpropionamide)-3-methylmaleamic acid), C12PMMA-PI) as a mixture of isomers (arising from the amidation and ring opening reaction occurring on either of the carbonyl groups of the cyclic anhydride). Further attempts to purify III using typical purification means led to a rapid hydrolysis of the maleamic acid and regeneration of PI. 300 MHz NMR (Dimethyl sulfoxide-d6, 99.5%, ppm) δ 8.64 (2H, dd, J=9.2, 8.2) 7.79-7.74 (5H, m) 7.56 (1H, dd, J=9.2, 2.3) 7.41-7.29 (2H, m) 7.24-7.14 (1H, m) 6.67-6.62 (1H, br m) 6.24 (1H, d, J=2.3) 6.02-5.96 (2H, br) 4.43-4.33 (2H, br m) 3.45-2.70 (21H, m) 2.40-1.85 (8H, m) 1.52-0.98 (46H, m) 0.87-0.80 (6H, m). In DMSO λₑₓ max=460, λₑₘ max=635.

C. Synthesis of non-rapidly reversible 3,8-bis-undecylcarbanylamino 5-(3,3-diethyl-3-methylammonium propyl) 6-phenyl phenanthridinium diiodide (IV, C12CON-PI, FIG. 1). Propidium iodide (36.1 mg, 0.0540 mmol, Aldrich) was taken up in dichloromethane (10 ml). Lauroyl chloride (26.9 μl, 0.113 mmol, 2.1 eq, Aldrich) was added to the solution under nitrogen, followed by diisopropylethylamine (20.7 μl, 0.119 mmol, 2.2 eq, Aldrich), and the resulting solution was stirred at ambient temperature (amidation of PI with lauroyl chloride). After 16 h, the solution was partitioned in EtOAc/H₂0, washed 2×H₂0, 1×brine, dried (Na₂SO₄), filtered, and concentrated. The resulting yellow solid was crystallized twice from acetonitrile to afford 53.7 mg (96%) IV. Molecular ion calculated for C₅₁H₇₈N₄O₂I=905.5, found m/e=905.7, molecular ion/2 calculated for C₅₁H₇₈N₄O₂=389.6, found m/e=389.9. 400 MHz 1H NMR (Dimethyl sulfoxide-d6, 99.5%, ppm) δ 11.80 (1H, s) 10.74 (1H, s) 9.16 (1H, s) 9.11 (1H, d, J=9.6) 9.06 (1H, d, J=9.2) 8.62 (1H, d, J=8.8) 8.48 (1H, dd, J=9.2, 2.0) 8.02 (1H, d, J=2.0) 7.85-7.74 (5H, m) 4.63-4.52 (2H, m) 3.33-3.25 (6H, m) 2.94 (3H, s) 2.42-2.28 (4H, m) 2.19-2.16 (2H, m) 1.68-1.45 (4H, m) 1.40-1.15 (38H, m) 0.85 (6H, t J=5.8). In H₂O λₑₓ max=443, λₑₘ max=546.

D. Synthesis of non-rapidly reversible 3,8-bis-dodecylamine 5-(3,3-diethyl-3-methyl-ammonium propyl) 6-phenyl phenanthridinium diiodide. (Propidium iodide bis-dodecylamine, C12-PI). 5-(3,3-diethyl-3-methylammonium propyl) 6-phenyl phenanthridinium diiodide PI (36.0 mg, 0.0539 mmol, Aldrich) together with molecular sieves (3 Å, 10 wt eq) were taken up in 2.7 mL EtOH. Laurinaldehyde (30 μL, 0.135 mmol, Fluka) was added and the solution was stirred at ambient temperature. After 24 h the solution was concentrated under reduced pressure and the resulting residue was dissolved in dichloromethane/acetonitrile (1:1, 10 mL), filtered, and concentrated under reduced atmosphere. The resulting solid was dissolved in dichloromethane/THF (1:1, 6 mL), sodium triacetoxyborohydride (22.8 mg, 2 eq, Aldrich) was added, and the resulting solution was stirred at ambient temperature. After 16 h, the solution was washed with NaOH (1×1N), H₂O, brine, dried (Na₂SO₄), filtered, and concentrated under reduced pressure to afford a deep purple solid. The residue was dissolved in dichloromethane (100 mL) and stirred rapidly under air to oxidize the pyridinium portion of the molecule. After 7 days, the solution was concentrated under reduced atmosphere and the residue was purified by reverse phase HPLC on a BetaBasic Cyano column (250×20, Keystone Scientific Inc.) to afford 13.9 mg of C12-PI (26%). Molecular ion/2 calculated for C₅₁H₈₂N₄=375.3, found m/e=375.5 400 MHz 1H NMR (Dimethyl sulfoxide-d6, 99.5%, ppm) δ 8.68 (1H, d, J=10) 8.64 (1H, d, J=10) 7.86-7.10 (9H, m) 4.72-4.45 (2H, br) 4.17-4.10 (2H, m) 3.32-3.08 (6H, m) 2.94-2.74 (7H, m) 2.34-2.16 (2H, m) 1.72-1.12 (46H, m) 0.90-0.79 (6H, m).

### Example 2

### Hydrophobic Modification of Melphalan Chemotherapeutic

A. Synthesis of rapidly reversible 3,9-diaza, 2-[4-{bis(2-chloroethyl)amino}phenyl methyl], 5-(2-carboxyl ethylidine), 4,8-dioxo uncosanoic acid, (V, C12PMMA-Melphalan, FIG. 1). To melphalan (18.2 mg, 0.596 μmol, Sigma) was added solid C12PMMA (see example 1; 41.9 mg, 0.119 mmol,), K₂CO₃ (82.4, 0.596 mmol), and molecular sieves (3 Å, 182 mg). To the resulting mixture was added anhydrous DMSO (1.82 ml), and the resulting suspension was heated at 60° C. for 12 h. The suspension was cooled to ambient temperature, centrifuged, and filtered (0.2 μm membrane) under inert atmosphere, to afford V (C12PMMA-Melphalan), as a mixture of isomers. Molecular ion+1 calculated for C₃₃H₅₁N₃O₆Cl₂=656.3, found m/e=655.5. Molecular ion−1 calculated for C₃₃H₅₁N₃O₆Cl₂=654.3, found m/e=636.3 (M-1-18). 400 MHz NMR (Dimethyl sulfoxide-d6, 99.5%, ppm) δ 7.94-7.72 (2H, m) 6.90 (2H, d, J_{AB}=8.6) 6.57 (2H, d, J_{AB}=8.6) 4.16 (1H, m) 3.72-3.50 (8H, m) 3.323-3.181 (2H, m) 2.99-2.96 (2H, m) 2.48-2.44 (0.8H, m) 2.21 (1.2H, t, J=7.4) 1.92 (1H, s) 1.81 (2H, s) 1.38-1.23 (20H, m) 0.85 (3H, t, J=6.8).

B. Synthesis of rapidly reversible DMODS-Melphalan (FIG. 2). By a similar procedure as in example 1, silylation of melphalan was conducted with chloro(dimethyl)-octadecylsilane (DMODSiCl) to afford DMODS-melphalan. Given the differences in functional groups present in melphalan, different labile modifications are possible. For example, melphalan has a tertiary amine, a primary amine, and a carboxylic acid. Given the reactivity of DMODSiCl, two modifications are expected to occur, one on the primary amine, and the second with the carboxylic acid to form a silylester. Analysis of the reaction by 1H NMR (250 MHz, N,N-dimethylformamide-d7) supported modification of the melphalan as described.

### Example 3

Hydrophobic modification of cisplatin chemotherapeutic. By a similar procedure as in example 1, silylation of cisplatin (cis-diamminedichloro-platinum(II), FIG. 2), was conducted with chloro(dimethyl)octadecylsilane (DMODSiCl) to afford Cl₂Pt(NH₂Si(CH₃)₂C₁₈H₃₇)₂ (BDMODS-CP). Analysis of the reaction (not optimized) of cisplatin and DMODSiCl by 1H NMR (250 MHz, N,N-dimethylformamide-d7) indicated the loss of the broad amine signal at δ4.2 (relative to TMS) and the appearance of the alkyl groups from the reaction with the chlorosilane. Integration of the relative signals indicated 1.7 alkyl groups per cisplatin molecule. This material was filtered through a 0.20 μm sterile Nylon filter.

### Example 4

### Hydrophobic Modification of Doxorubicin Chemotherapeutic

A. Synthesis of DMODS-Doxorubicin (FIG. 2). Doxorubicin HCl (2.00 mg, 0.00345 mmol, Aldrich) was taken up in 200 μL of DMSO. To the resulting solution was added molecular sieves (3 Å, 20 mg), K₂CO₃ (4.8, 0.035 mmol), and chloro(dimethyl)-octadecylsilane (2.4 mg, 0.0069 mmol, Aldrich). The reaction was stirred at ambient temperature. After 16 h, the resulting blue solution was diluted with DMSO (200 μL) and centrifuged to remove solids to afford DMODS-Doxorubicin.

B. Synthesis of C12PMMA-Doxorubicin. Doxorubicin HCl (2.00 mg, 0.00345 mmol, Aldrich) was taken up in 200 μL of DMSO. To the resulting solution was added molecular sieves (3 Å, 20 mg), K₂CO₃ (4.8, 0.035 mmol), and C12PMMA (2.4 mg, 0.0069 mmol). The reaction was stirred at ambient temperature. After 16 h, the resulting blue solution was diluted with DMSO (200 μL) and centrifuged to remove solids to afford C12PMMA-Doxorubicin.

### Example 5

Development of a Mixing Chamber. Due to the instability of the RRH-PI prodrugs in water, it was necessary to dissolve them in a small amount of pharmaceutically acceptable organic solvent (OS) and mix them with an aqueous solution immediately prior to delivery. Rapid and efficient mixing was critical for optimum delivery and minimalization of toxicity due to the organic solvent. A passive mixing chamber with colliding flows was utilized to mix the drug/RRH-prodrug (dissolved in a carrier solvent such as an organic solvent) with an aqueous solution immediately prior use. Two syringe pumps (Harvard Pumps, PHD 2000) were utilized to deliver the solutions to the mixing chamber. Typically mixtures were 1:10 by volume with 0.67 μl of PI or RRH-PI/OS solution (7.48 mM) diluted with 6.7 μl of isotonic glucose (ITG), buffer, or media per sec, for a total delivery volume of 220 μl (0.150 μmol of PI or RRH-PI) over 30 sec (final organic solvent concentration of 9.1% by vol.).

The mixing chamber (FIG. 3) was constructed from 18 G stainless steel tubing (38 mm in length) by drilling a 0.2 mm hole at a 45 degree angle in the tubing wall and then inserting 30 G stainless steel tubing. The 30 G stainless tubing was angled and advanced into the 18 G tubing so as to be centered within the larger tubing and then soldered in place. Tubing connectors were attached to the chamber inlets by successively soldering 23 G and 27 G stainless steel tubing in place. Polyethylene tubing (PE10 Intramedic tubing, Becton Dickinson and Company) was attached to the chamber inlets for connection to the syringes. Drug or prodrug in organic solvent and aqueous solution (1:10 by volume) were pumped in a colliding direction, creating a turbulent mixing flow

Results: Testing with this mixing chamber (PI in DMF and isotonic glucose) indicated very little PI uptake in cells, and was comparable to control experiments in which an aqueous solution of PI (no OS) was added to the cell media. This indicated that the mixing chamber was effective at mixing the solutions, resulting in no cellular PI uptake due to OS mediated toxicity. This mixing chamber was utilized in all of the described experiments.

### Example 6

Analysis of prodrug lability. The prodrugs were tested for their rate of hydrolysis at pH 6.0, 7.2 and 8.5 using fluorescent spectroscopy. Lability studies for C12CON-PI and RRH-PI (rapidly reversible hydrophobic-PI) derivatives were conducted on a Cary Eclipse Fluorescence Spectrophotometer (Varian Inc.).

To solutions of PI and RRH-prodrugs of PI (7.48 μM in DMSO) were added various amounts of buffer (25 μL, 50 μL, and 100 μL of 20 mM Hepes buffer) at different pH. Upon cleavage of the hydrophobic modification and regeneration of PI, the fluorescence of the solution increased (λₑₓ=493 nm, λₑₘ=647 nm, excitation and emission slit width=5 nm, and emission PMT=600V) and was assayed as a function of time (20 min).

Alternatively, to solutions of fluorescamine (857 μM in DMSO) were added various amounts of buffer (20 μL, 30 μL, 40 μL, 50 μL, and 70 μL of 100 mM MES buffer, pH 6.0) followed by melphalan or V (249 μM final concentration in DMSO). The fluorescence of the solution (λₑₓ=380 nm, λₑₘ=464 nm, excitation and emission slit width=5 nm, and emission PMT=600V) was assayed as a function of time (40 min). At each buffer concentration the reaction of fluorescamine and melphalan was more rapid than hydrolytic breakdown of V.

The resulting curves were normalized for intensity and fit to an exponential equation (Origin) to determine k_{determined} for each solution. The k_{determined} for each compound at appropriate pH were plotted against the mol % of H₂O, and the best fit line was solved for k_{buffer} for 100 mol % H₂O. These derived k_{buffer} values were used to determine the half-life of the prodrug in pure buffer according to the equation t_{1/2}=1n2/k.

Hydrophobization of PI resulted in a shift in the excitation maximum and a decrease in the measured fluorescence of the RRH-PI. Upon the addition of aqueous buffer to II or III, there was a rapid increase in fluorescence, indicating the loss of the hydrophobic group and reversion of the RRH-PI to unmodified PI. The silazane II was hydrolytically unstable with a calculated t_{1/2} of 23 sec at pH 8.5. As the pH of the buffer was decreased to pH 7.2, the rate of hydrolysis increased (t_{1/2}=11 sec). A similar trend was observed for III, with t_{1/2}'s of 21 sec (pH 8.5) and 8 sec (pH 7.2). Hydrolysis of V was monitored by the reaction of the liberated melphalan with fluorescamine, resulting in a t_{1/2} of 28 sec at pH 6.0.

TABLE 1
Half-life Values for hydrophobic-PI, RRH-PI and Melphalan prodrugs.

| | Half-life (k) | | |
|---|---|---|---|
| Compound | pH 8.5 | pH 7.2 | pH 6.0 |
| BDMODS-PI (II) | 23.1 sec | 10.5 sec | |
| | (1.80 min⁻¹) | (3.96 min⁻¹) | |
| C12PMMA-PI (III) | 21.1 sec | 8.24 | |
| | (1.97 min⁻¹) | (5.05 min⁻¹) | |
| C12CON-PI (IV) | non-labile | non-labile | |
| C12PMMA-Melphalan | | | 25.7 sec |
| (V) | | | (1.62 min⁻¹) |

### Example 7

In Vitro cellular uptake of PI, RRH-PI compounds, and IV evaluated by fluorescence measurements. PI and the RRH-prodrugs were tested for cellular uptake on several cell lines which included B16 (murine melanoma), Hepa 1-6 (mouse hepatoma), SK-OV-3 (also SKOV-3, human ovarian carcinoma), OVCAR-3 (human ovarian carcinoma), Jurkat (human T-lymphocyte), 293 (human embryonic kidney), and MC38 (mouse colon carcinoma) cells. Adherent cells were plated at 2.5×10⁵ cells/well on 6-well plates 24 h prior to testing. The growth media was removed from individual wells and the drug/prodrug was immediately added to the well with the mixing chamber. Final drug/prodrug concentrations were 0.150 μmol in 220 μl total volume (20 μl OS, 200 μl ITG). After 30 sec the drug solution was removed (aspirated) and 2 ml complete media was added to the cells. Experiments conducted with prodrug that was hydrolyzed prior to application to the cells followed a similar protocol, however the combined solution obtained after passing through the mixing chamber was collected and allowed to sit at ambient temperature for 5 min prior to application on the cells. Experiments conducted with Calcein AM (Invitrogen, 100 μl of 20 μM solution in complete media) followed a similar protocol for drug treatment (4 min treatment). Experiments conducted with SYTOX Green (Invitrogen) followed a similar protocol for drug treatment. SYTOX Green (20 μl of 5 μM solution in 20 mM Hepes, pH 7.4) was added to the wells and the cells were examined. Each condition was repeated in 2 or 3 wells, and the cells were immediately examined by microscopy. Fluorescent microscopy studies were conducted with a three laser LSM 510 confocal microscope, an Axiovert S100 fluorescent microscope, or an Axioplan2 fluorescent microscope (all Zeiss) equipped with filter sets for the rhodamine fluorescence channel (λex=BP 450/12, λem=LP 590 nm filter set) and the fluorescein fluorescent channel (λex=BP 546/90, λem=LP 515 nm filter set). Imaging was performed with an AxioCam digital camera (Zeiss), with AxioVision imaging software (V 4.5, Zeiss). For all images, identical camera settings and exposure times were used on comparative sample and control images. Images were processed with Photoshop (Adobe) for level and brightness using identical settings between sample and control images.

For experiments conducted with IV, the cells were plated on 6-well plates with cover slips. Following treatment as above, the cover slip was immediately removed and inverted over 1 drop of media for imaging, or the cells were incubated for 1 h prior to removal of the cover slip and imaging. Imaging was conducted on an Axioplan2 fluorescent microscope (Zeiss) and the same fields were imaged with phase contrast illumination, in the rhodamine fluorescence channel (λₑₓ=BP 450/12, λₑₘ=LP 590 nm filter set), and in the fluorescein fluorescent channel (λₑₓ=BP 546/90, λₑₘ=LP 515 nm filter set). IV was also mixed with PI and then delivered to SK-OV-3 cells, and the cells were imaged as before. In one protocol, equal amounts of IV (15.0 mM in DMSO) and PI (15.0 mM in DMSO) were combined and the resulting solution was added to cells via the mixing chamber. In a second method, PI was taken up in the ITG solution (0.748 mM) and mixed with IV (7.48 mM in DMSO) in the mixing chamber. Similarly, V (15 mM and 30 mM in DMSO) was mixed with PI and delivered to Hepa 1-6 and SK-OV-3 cells using the second method. The cells were imaged as before following 30 sec-4 min exposure to the drug solution.

For experiments conducted on Jurkat cells (8.0×10⁵ cells/ml in media), the cells were isolated by centrifugation and resuspended in ITG at a density of 4.0×10⁶ cells/ml. Exposure to the drug/prodrug was conducted by passing the cells in suspension in ITG (200 μl) through the mixing chamber. After 30 sec the cells were centrifuged and the drug solution was removed (aspirated), the cells were resuspended in complete media (2 ml) and plated in 6-well plates for imaging. Passing the cells through the mixing chamber (no drug/prodrug treatment) had no effect on cell viability when compared to cells plated without passing through the mixing chamber.

Flow cytometry was conducted on a FacsCalibur Flow Cytometer (Becton Dickinson Biosciences). Jurkat cells were isolated and treated with PI and II as previously described. After 4 min the cells were centrifuged and the drug solution was removed (aspirated) and 1 ml complete media was added to the cells. During analysis, PI was added to control cells (1 ml of 8.0×10⁵ cells/ml in complete media, 1 drop of 1 mg/ml PI solution, no OS or mixing chamber) to determine normal cell senescence. Data were collected for a total of 10,000 events using Cell Quest™ (Becton Dickinson Biosciences), and analyzed with FlowJo (Tree Star Inc.) for single cell events (approximately 80% of events), and secondly for PI. For Jurkat cells treated with II, forward scatter was analogous to control cells, while side scatter increased approximately two fold. Reported data represents a histogram of PI intensity for single cell events from four independent preparations. Cells were considered PI positive at a value of 100 on the PI intensity axis of the histogram for the cell percentages described.

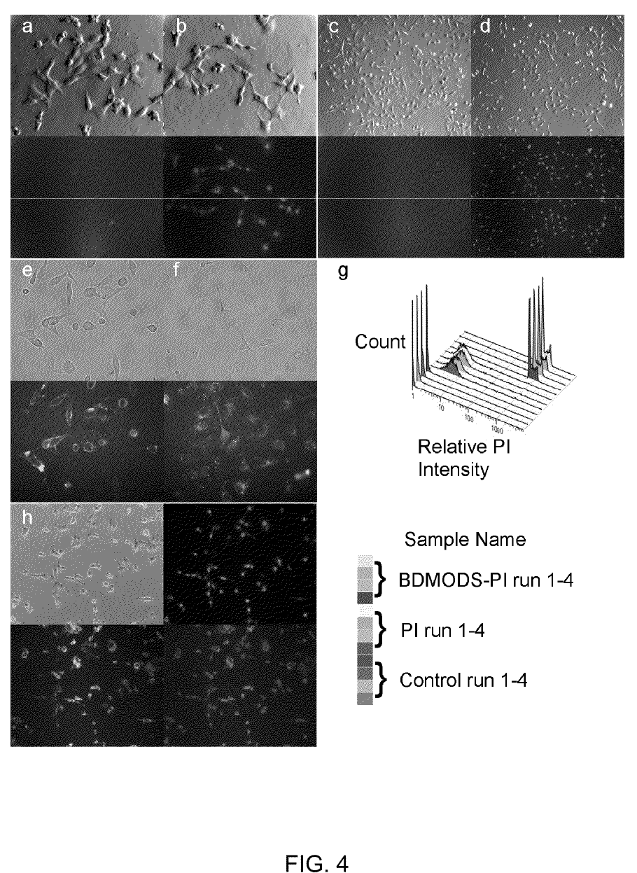

Results: PI, RRH-PI prodrugs, and IV were tested for the ability to stain viable cells (both tumor cells and lymphocytes) which included B16 (murine melanoma), Hepa 1-6 (mouse hepatoma), SK-OV-3 (human ovarian carcinoma), OVCAR-3 (human ovarian carcinoma), Jurkat (human T-lymphocyte), 293 (human embryonic kidney), and MC38 (mouse colon carcinoma) cells. Stained cells is indicative of successful PI-prodrug uptake, intracellular release of functional PI, and DNA intercalation. FIG. 4a-b show representative results following the application of PI and II to Hepa 1-6 cells. Unmodified PI stained very few cells (FIG. 4a), while II yielded strong nuclear staining in all of the cells (FIG. 4b). Using longer exposure times, fluorescent signal could also be detected within the membrane and cytoplasm of the cells exposed to II (cytoplasmic PI is not as fluorescent as the nuclear, DNA-intercalated, PI). Phase contrast microscopic examination indicated that the cells that took up II appeared to be morphologically intact. Similar results were observed following the treatment of SK-OV-3 cells (FIG. 4c-d). PI treatment resulted in a small percentage of PI positive cells (FIG. 4c), while treatment with II resulted in nearly all of the cells being PI positive (FIG. 4d). II that was premixed with ITG (5 min) before application to the cells, resulted in very few PI positive cells (identical to wells receiving PI treatment), indicating prodrug hydrolysis resulting in the formation of native PI (data not shown).

The RRH-prodrug III was similarly investigated for cellular uptake. In all cases, the results with III paralleled the results obtained with II. Additionally, results from the application of PI, II, and III on other cell lines (OVCAR-3, Jurkat, 293, and MC38) were similar to those detailed for the Hepa and SK-OV-3 cell lines indicating that internalization of the RRH-PI was not cell type specific.

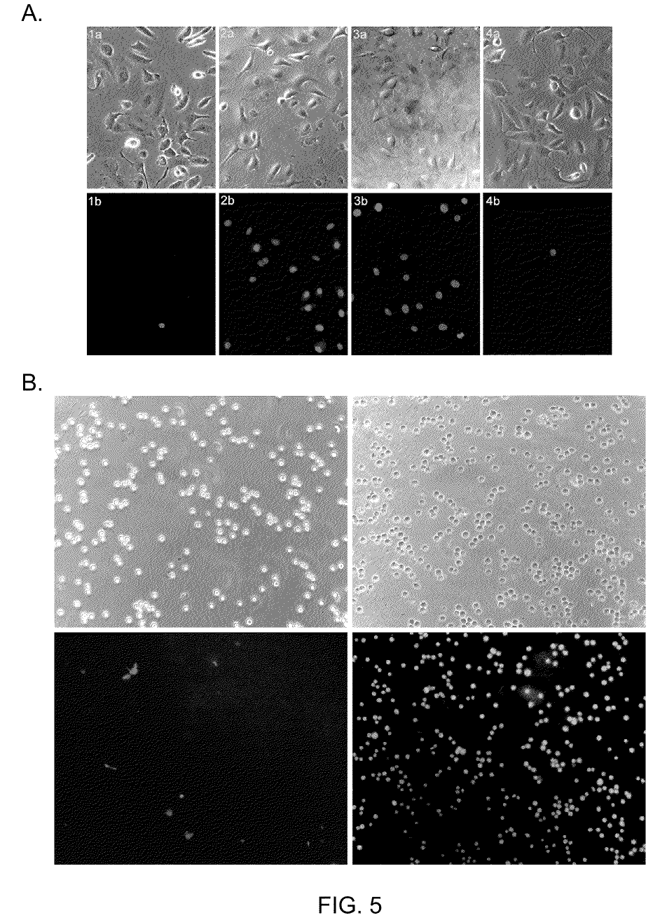

FIG. 5A-B show additional results from the addition of PI, RRH-PI prodrugs, or hydrolyzed PI-prodrug to SK-OV-3 (FIG. 5A) or Jurkat (FIG. 5B) cells. In SK-OV-3 cells, unmodified propidium iodide stained very few cells (FIG. 5A, panel 1), representing normally occurring dead cells in the population. BDMODS-PI and C12PMMA-PI stained 60-80% of the cells (FIG. 5A, panels 2-3), demonstrating that the hydrophobically modified prodrugs efficiently enter viable human ovarian cancer cells with successful intracellular formation of active free propidium iodide. Premixing of BDMODS-PI in ITG, which permits hydrolysis of the labile linkage and release of propidium iodide did not show PI positive staining (FIG. 5A, panel 4b). In the case of Jurkat cells (human T-lymphocyte), unmodified propidium iodide stained very few cells, while C12PMMA-PI exhibited strong cellular uptake and staining (essentially 100% of the cells, FIG. 5).

Flow cytometry was used to quantitate prodrug delivery to Jurkat cells. Flow cytometry of cell suspensions that were treated with PI in aqueous solution (no OS, no mixing chamber) indicated very low PI uptake in most cells, with very few displaying a signal above 100 (0.3±0.1%, control run 1-4, FIG. 4g). For cells that were suspended in ITG, and passed through the mixing chamber with unmodified PI (in DMSO), few PI positive cells were observed (8.4±0.6%, PI run 1-4). Flow cytometry on cells that were suspended in ITG, and passed through the mixing chamber with II in DMSO indicated that nearly all cells were PI positive (99.5±0.1%, BDMODS-PI run 1-4). The results from the flow cytometry correlated well with our observations using fluorescent microscopy for cells treated with RRH-PI prodrugs.

The stable PI derivative, IV, was also tested for cellular uptake in SK-OV-3 cells (FIG. 4e-f). Immediately following application of IV, a diffuse signal was observed along the cell membrane in the fluorescein channel (FIG. 4e). After one hour incubation, a more defined punctate signal was observed (FIG. 4f), likely due to endocytosis of the membrane-bound derivative. Thus, in the absence of lability, the hydrophobic prodrug was sequestered and retained in the membrane.

In order to investigate whether the RRH-PI prodrug was causing membrane damage or acute cellular toxicity by virtue of its amphipathicity, possibly resulting in drug internalization by diffusion through compromised cell membranes, several different studies were conducted. Calcein AM was used as a live cell marker in Hepa 1-6 cells following treatment with II (FIG. 4h). Calcein AM is a cell permeable non-fluorescent dye that is converted to fluorescent calcein by intracellular esterases. Following cellular treatment with II, approximately 86% of the cells were both PI and calcein positive, indicating viable cells. In another experiment, SYTOX Green, a dead cell indicator, was added to cells following treatment with II. With this indicator, less than 5% of cells were both PI-positive and SYTOX Green positive (data not shown).

Additional studies were conducted in which the stable PI derivative IV was tested together with PI for cellular uptake in SK-OV-3 cells. Two different methods were employed, mixing PI with IV in DMSO or including PI in the aqueous diluting solution (ITG). Both methods yielded similar results, with no increased levels of nuclear PI staining detected as compared to cells treated with PI alone (data not shown). Similarly, in a series of experiments conducted by mixing the melphalan derivative V with PI and testing for cellular uptake, no increased levels of nuclear PI staining were observed in either Hepa 1-6 and SK-OV-3 cells (data not shown). In addition, a trypan blue assay for membrane disruption also indicated that neither V or methyl aniline silylated with chloro(dimethyl)octadecylsilane caused generalized membrane disruption (data not shown). Therefore, neither the RRH-prodrug nor its stable analogue facilitated delivery of another molecule not linked to the hydrophobic group. These results suggest that internalization of the PI moiety of RRH-PI derivatives is not due to generalized membrane damage.

### Example 8

Antiproliferative/Cytotoxic in vitro studies on PI, RRH-PI prodrugs, melphalan, and C12PMMA-melphalan. In vitro cytotoxicity testing was conducted on Hepa 1-6 (mouse hepatoma), SK-OV-3 (human ovarian carcinoma), and MC38 (mouse colon carcinoma) cells using a tetrazolium based assay (WST, Dojindo Molecular Technologies). For the WST assay, Hepa 1-6 (2.0×10⁵ cells well), SK-OV-3 (7.5×10⁵ cells well), and MC38 (8.5×10⁵ cells well) were seeded in 1000 μL media into 12-well plates 24 h prior to testing (starting confluency ˜50%). Following removal of media, drug/prodrug solution was added drop-wise to quadruplicate wells, using the mixing chamber. Effective drug concentrations were calculated from the amount of drug added in the total volume of OS and aqueous solution. Following 4 min of drug solution exposure, the solution was removed, and 1000 μl fresh complete media was added to each well. After the cells were incubated for 24-48 h, WST-1 (20 μl of a 5 mM solution in PBS) and N-methylphenazonium methyl sulfate (PMS, 20 μl 0.2 mM solution in PBS) were added and the cells were incubated for 1-4 h. For testing with MC38 cells, the amounts of WST-1 and PMS were doubled. Following incubation, 100 μl of sample was transferred to quadruplicate wells on a 96-well plate, and the absorbance (438 nm) values were measured on a SPECTRAmax Plus³⁸⁴ microplate spectrophotometer (Molecular Devices Corporation). Data represents the mean A438 values of 16 wells with standard deviation, corrected for media contribution, and normalized against cells in media with no drug/prodrug treatment (reported as % cell viability±standard deviation).

The concentration of drug (μM) that is required for 50% inhibition in vitro is reported as the IC₅₀. The reported values were calculated from the best fit line (Excel) for a plot of effective drug concentration against % cell viability, and include the r² value. The reported IC₅₀ values for multiple RRH-PI plots are reported as IC₅₀ (μM)±standard deviation.

Results: Two days following a four min exposure of the cells to varying concentrations of V in DMSO, the cultures were tested for viability using a tetrazolium based assay. In Hepa 1-6 cells, the WST-1 cell viability assay indicated an IC₅₀ of 330 μM (r²=0.94) (Table 2). Treatment with melphalan had a slight effect on cell viability, with 81% (±2.3%) cell viability at the highest concentration tested (1458 μM). Previous studies have observed that lower concentrations of melphalan caused higher cellular toxicity, but our experiments used shorter drug exposure times (4 min v. 48 h) in order to model first pass exposure. No toxicity was observed from the DMSO/ITG vehicle alone. V that was premixed with ITG containing 20 mM Hepes buffer, pH 6.5, (10 min) to hydrolyze the prodrug before application to the cells resulted in cell viability levels similar to those observed with melphalan.

In SK-OV-3 cells, a similar toxicity profile was observed, with V showing much greater toxicity (IC₅₀=384 μM, r²=0.92) relative to melphalan (70±11% cell viability at the highest tested dose of 1458 μM, Table 2). Additionally, in MC38 cells, the WST-1 assay indicated an IC₅₀ of 304 μM (r²=0.93) for V and an IC₅₀=1176 μM (r²=0.91) for melphalan.

Similar cell proliferation/toxicity studies were done using RRH-PI compounds (Table 2). Although PI intercalates into DNA and would therefore interfere with DNA replication, its cell impermeability causes it to have little cellular toxicity. By forming a RRH-PI prodrug we were able to convert PI into a cytotoxic agent. In Hepa 1-6 cells, the WST cell viability assay at 24 h post treatment indicated an IC₅₀ of 282 μM (±62 μM) for II (Table 3). PI had little effect on cell viability, with 100% (±3.0%) cell viability at the highest concentration tested (1.25 mM). The WST-1 cell viability assay at 48 h post treatment indicated an IC₅₀ of 334 μM (±72 μM) for II, while PI again had little effect on cell viability at the highest concentration tested (93±5.7% cell viability at 1.7 mM). II that was premixed with ITG (10 min) before application cells resulted in levels of cell viability that were similar to the PI treated cells (108±1.3% at 833 μM, the highest concentration tested). The stable PI derivative IV was also tested for toxicity in Hepa 1-6 cells at 24 and 48 h post treatment, resulting in IC₅₀'s of 585 (±279 μM) and 429 μM (±37 μM) respectively. The toxicity resulting from IV may have been the result of endocytosis of the prodrug (hydrolysis of the amide would then yield PI), or its cationic amphipathic property. Regardless, the rapidly reversible prodrug II was more cytotoxic than IV, and would be less likely to cause cellular toxicity of non-targeted cells. Similar results were obtained with SK-OV-3 cells (Table 3).

TABLE 2
Mean IC₅₀ values (μM, std. dev.) for Drug/Prodrug in various cell
cultures using the WST Assay 24 and 48 h after drug exposure.

| | Mel- | | | | | | | |
|---|---|---|---|---|---|---|---|---|
| Cell | phalan | V | PI | | II | | IV | |
| Type | 48 h | 48 h | 24 h | 48 h | 24 h | 48 h | 24 h | 48 h |
| Hepa | >1458^{a} | 330 | >1,250^{b} | >1,700^{b} | 282 | 334 | 585 | 429 |
| 1-6 | | (93) | | | (62) | (72) | (279) | (37) |
| SK- | >1458^{c} | 384 | >1,250^{b} | >1,700^{b} | 194 | 257 | 455 | 342 |
| OV-3 | | (97) | | | (143) | (54) | (126) | (27) |
| MC38 | 1176 | 304 | | | | | | |
| | (150) | (94) | | | | | | |

^{a}Cell viability was ~80% of no treatment control at these maximum concentrations.
^{b}Cell viability was >90% of no treatment control at these maximum concentrations.
^{c}Cell viability was ~70% of no treatment control at these maximum concentrations.
Cell viability was >90% in cultures exposed to the vehicle alone (DMSO/ITG).

### Example 9

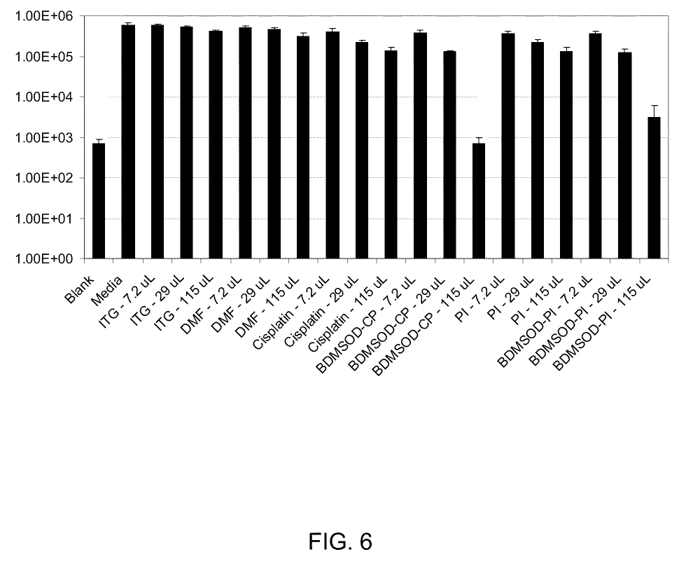

Enhanced antiproliferative/cytotoxic effect of RRH-PI and RRH-cisplatin prodrugs on B16 urine melanoma cells. To determine whether hydrophobically modified drugs demonstrate enhanced antitumor activity, we performed in vitro cytotoxicity testing on B16 murine melanoma cells. Using the dual pump colliding flow mixing chamber delivery system for drug delivery, we evaluated the effect of propidium iodide, BDMODS-PI, cisplatin, and BDMODS-cisplatin (BDMODS-CP) on B16 cells using the CellTiter-Glo luminescent cell viability assay (FIG. 6). Cells were seeded at 1×10⁴ cells/well in 100 μl of media into 96-well plates on day 0 and cultured for 24 h prior to addition of drug. Following removal of media, drug solution was added drop-wise (1, 4, or 16 drops; with effective delivery of 7, 24, and 112 μl, respectively, of solution) to quadruplicate wells, using a 1:11 DMF/ITG solution ratio with the dual pump mixing chamber. Following 10 min of drug solution exposure, 100 μl of fresh media was added to each well and incubated for 3 h. Drug-containing media was replaced with 100 μl of fresh media and cultured for and additional 24 h. Data represent the mean RLU values of quadruplicate wells ±S.D. Drug concentrations evaluated: cisplatin (2.5 μg/μl DMF), propidium iodide (5.0 μg/μl DMF).

Results: The ITG carrier solution, as well as DMF, exhibited negligible antiproliferative effects against B16 cells when compared to media only controls. Cisplatin showed a mild dose-dependent antiproliferative response, with maximal effect at the highest drug level tested (24 μg drug in 112 μL of DMF/ITG delivery solution per well). In comparison, the modified cisplatin prodrug markedly enhanced the antiproliferative and/or cytotoxic activity of the drug. At the highest level tested, BDMODS-CP reduced RLU levels to those observed with blank wells (media only wells without B16 cells), indicating complete cytotoxic effect against the B16 tumor cells. Similar trends were observed for PI and BDMODS-PI. These results clearly indicate that hydrophobic modifications of PI, cisplatin, and melphalan facilitates enhanced antitumor effects against melanoma and colon carcinoma cells in vitro.

### Example 10

Light Scattering analysis of prodrugs. Particle size was determined by dynamic light scattering on a Zeta Plus (Zeta Potential Analyzer, Brookhaven Instrument Corporation, λ 533 nm, 90°), and results are reported as the size of the most abundant particle (range of particle sizes, counts sec⁻¹). Fluorescent compounds were also analyzed for particles qualitatively, by measuring for light scattering on a Cary Eclipse Fluorescence Spectrophotometer (Varian Inc.), with λₑₓ=λₑₘ (300-800 nm, 90° detection). The presence of particles in solution results in an increase in signal detection at 90°. PI and RRH-PI prodrugs were 7.48 mM in DMSO. Melphalan and V were 50 mM in DMSO. The drug/prodrug solutions were mixed and diluted with ITG through the mixing chamber (40 μl OS and 400 μl ITG), and analyzed immediately.

Results: Analysis of the stable prodrug IV (following mixing with ITG) by dynamic light scattering, indicated the formation of 36 nm particles (range=28-42 nm, 18.3 Mcps). Similarly, dynamic light scattering analysis of V (following mixing with ITG) also indicated the formation of 36 nm particles (range=36-66 nm, 2.1 Mcps). Instability in aqueous solution and absorption of laser light (λ 533 nm) prevented direct measurement of RRH-PI prodrugs II and III by dynamic light scattering. Samples were therefore analyzed qualitatively for the presence of particles by measuring the 90° light scattering. Examination of IV (positive control) indicated a 15-fold increase in signal intensity relative to ITG or PI in ITG. However, the analysis of II and III by 90° light scattering indicated a maximum two fold increase in signal intensity relative to PI. Based on these results, we cannot definitively establish the presence of RRH-PI prodrugs micelles. Given that the stable derivative IV and the melphalan derivative V did indicate the presence of micelles, it is probable that micelles do form for the RRH-PI prodrugs, although transient in nature due to the rapid lability of the prodrug.

### Example 11

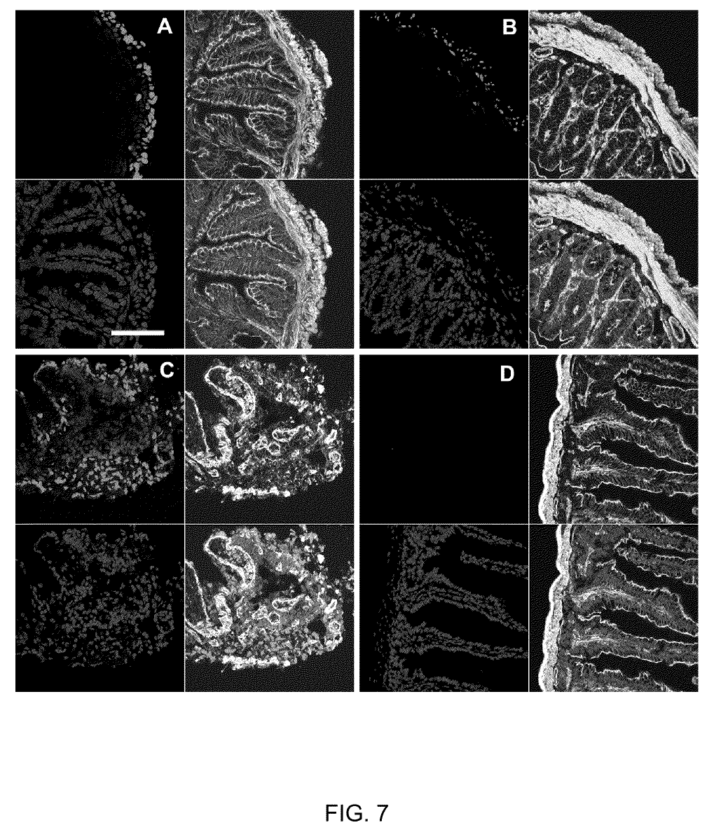

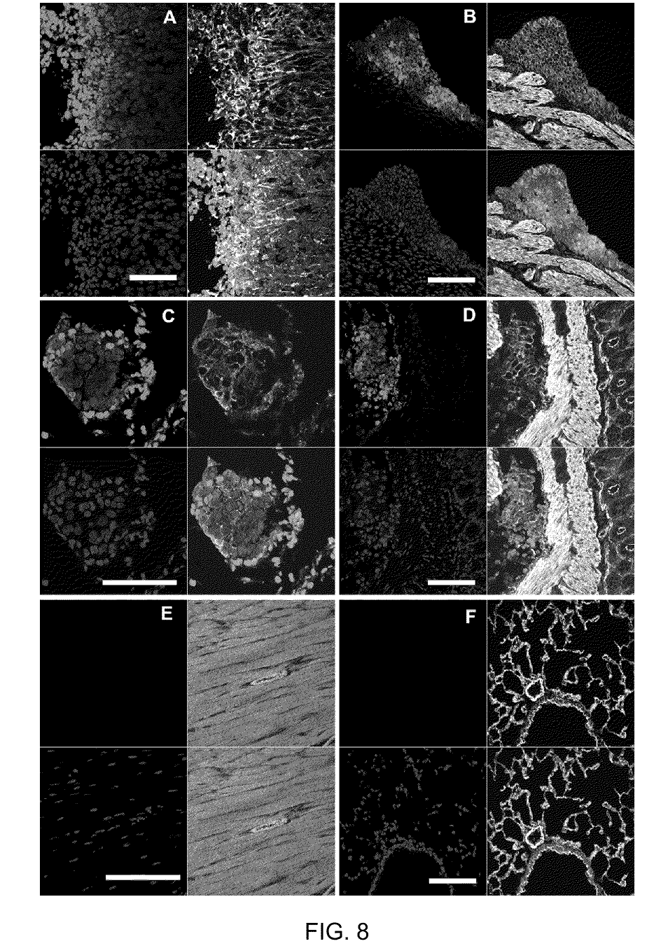

BDMODS-PI and C12PMMA-PI show enhanced drug uptake by surface tissue following IP application. For evaluation of prodrug delivery to exposed tissues in the peritoneum, we tested IP application of propidium iodide, BDMSODS-PI, and C12PMMA-PI to both normal mice and in a mouse model of disseminated peritoneal ovarian cancer. All procedures were executed under Isoflurane inhalation anesthesia. In normal mice either the abdominal cavity was opened and the drug mixture was directly applied on abdominal organs (220 μL of drug-OS/ITG over 30 sec), or the drug mixture was injected through the abdominal wall (1 mL of drug-OS/ITG over 1 min). For both delivery protocols, the dual pump mixing chamber was used. After 10-60 min, the animals were euthanized and tissues were harvested, frozen, sectioned, stained with ToPro-3 and Phalloidin Alexa 488, and examined by laser confocal microscopy. Application of propidium iodide in normal mice resulted in extremely rare nuclear labeling (data not shown), while application of both BDMODS-PI, and C12PMMA-PI resulted in near-exclusive PI⁺-staining of cells exposed to the peritoneal cavity. The cells situated deeper in the tissues, appeared to be labeled at a lower intensity (FIG. 7-8).

### Example 12

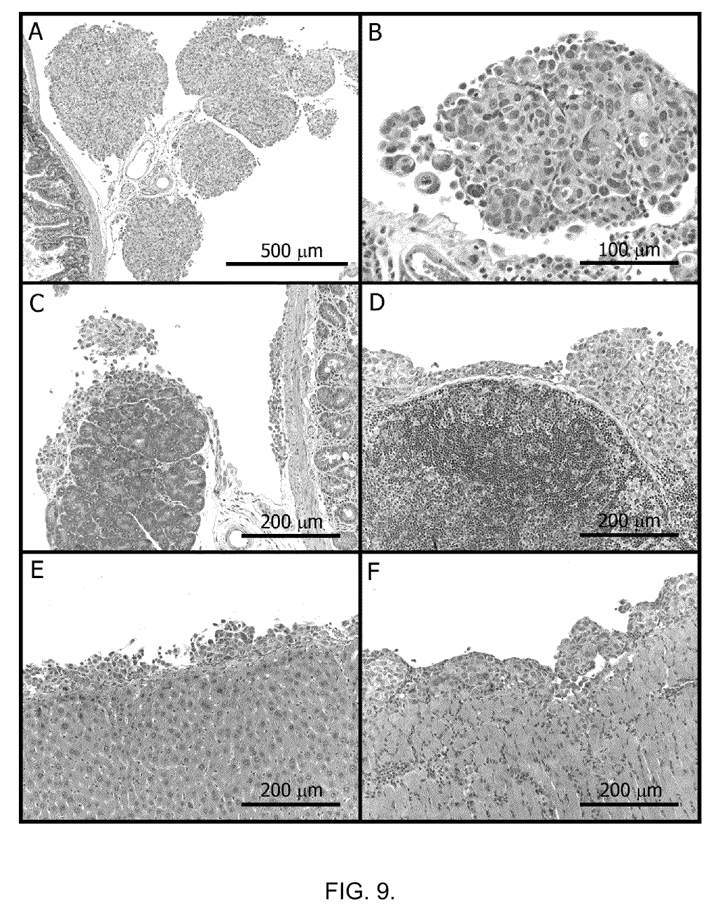

BDMODS-PI and C12PMMA-PI show enhanced drug uptake by surface tissue and microtumors following IP and intraperitoneal perfusion chemotherapy (IPPC) application in a mouse tumor model. For establishment of the cancer model, 2×10⁶ SK-OV-3 cells were injected IP into nude mice. The mice were examined at two weeks following cell inoculation, or at the first manifestation of ascites (about 4-5 wks). Tissue samples were fixed in 10% NBF, routinely processed, stained with H&E stain, and subjected to histopathological analysis. Microscopic examination indicated that at two weeks after SK-OV-3 cell inoculation, multiple microtumors (about 1 mm) were present throughout the peritoneal cavity, most notable on the mesentery. At 4-5 weeks after SK-OV-3 cell inoculation, maximum tumor size increased to 5-7 mm with the bulk of cancer development present as about 0.1 to about 1 mm microtumors (FIG. 9). At 5 wks most peritoneal surfaces were affected by growing cancer cells, coating both the visceral and parietal peritoneum, e.g. liver, pancreas, and diaphragm. Thus, the histopathological analysis indicated strong similarities in peritoneal perpetuation and dissemination between the SK-OV-3 mouse model and clinical ovarian cancer.

Two weeks following cell inoculation, the abdominal cavity was opened and the drug mixture was directly applied on the duodenum and ovary/uterus/fallopian tubes (220 μL of drug-OS/ITG over 30 sec). Alternatively, the drug mixture was injected through the abdominal wall (1 mL of drug-OS/ITG over 30 sec) as previously described. Both delivery routes utilized the described mixing chamber. After 10-60 min, animals were euthanized, tissue sections were harvested, and frozen sections were stained with ToPro-3 and Phalloidin Alexa 488, and examined by laser confocal microscopy. Direct application and the IP injection of both BDMODS-PI and C12PMMA-PI resulted in similar observations. Cellular uptake of the prodrug was stronger at the surface of the metastases, but also readily detectable in the middle of the small tumors (tumor size about 0.5 to about 1 mm; FIG. 7-8). In addition to labeling relatively large tumors, smaller tumors (about 25-100 cells in cross-section), growing on the visceral peritoneum and mesentery were also intensely labeled with BDMODS-PI and C12PMMA-PI. Adjacent mesentery cells indicated prodrug uptake and staining, as did the outer layer of cells of most of the normal tissues exposed to the peritoneal cavity and the prodrug solution (e.g., liver). However, the tumor lesions appeared to be more susceptible to prodrug uptake, showing greater tissue penetration and intense PI⁺-staining as compared to non-malignant tissues. Extensive sectioning and analysis indicated that tumors of any size were effectively targeted (up to 500 μm from the tumor surface). Tumors without propidium iodide-staining were not observed. In normal mice controls, similar analyses indicated near-exclusive PI⁺-staining of the outer cells exposed to the peritoneal cavity. Cells situated deeper in the tissues were labeled at a much lower intensity or not at all.

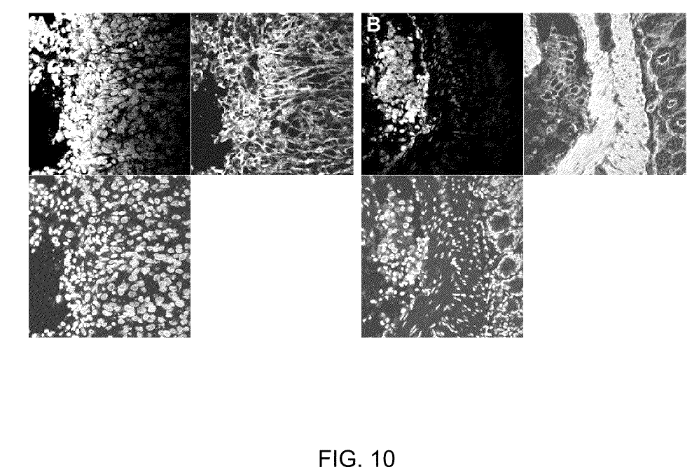

In a separate experiment, ovarian cancer development was monitored until signs of ascitis (4-5 wks). At this time, a test IPPC injection was conducted with C12PMMA-PI by adapting methodology typically used for peritoneal perfusion. Briefly, two 23 G Abbocath-T effluent catheters with multiple perforations were inserted into the peritoneal cavity and advanced to the region of the ovaries on both sides of the vertebra. The ascitic fluid was slowly aspirated with minimal negative pressure. Then an additional 23 G perforated catheter was inserted into abdomen and positioned on top of abdominal organs. 1 mL of drug/DMF/ITG solution was infused over 1 min, followed by repeated gentle massage. 5-7 min post administration, peritoneal fluid was again aspirated, and the peritoneal cavity was perfused with 10 mL of PBS via the top catheter, together with simultaneous aspiration via the lower two catheters. Special care was taken to avoid elevated abdominal pressure during the procedure. All animals survived the perfusion well and were sacrificed 3-5 h later. Confocal microscopy indicated a similar staining pattern as observed previously, with strong propidium iodide nuclear labeling of all of the outer cells exposed to peritoneal cavity, including ovarian tumors. Large tumors (5-7 mm) were labeled to a depth of about 500 microns (FIG. 10A), while all tumor cells in microtumors (0.1-1 mm) were heavily labeled throughout the tumor (FIG. 10B). All cells exposed to peritoneal cavity and thereby to the prodrug solution indicated prodrug uptake and staining, including: mesentery cells, outer layer of cells of abdominal organs, and disseminated peritoneal ovarian tumors. The tumor lesions still appeared to be more susceptible to drug uptake, indicating both greater tissue penetration and intense PI⁺-staining as compared to normal abdominal tissue, with the exception of the mesentery which was also was heavily PI⁺.

Generally, tissue penetration of small molecular weight drugs is difficult to accomplish. Several studies have indicated tissue penetration depths (many tumor types) for a variety of anti-neoplastics on the order of hours to days for 50-500 μm penetration. In contrast, our prodrugs resulted in a 500 μm penetration depth within 10 min. This could be a result a more effective interaction of the hydrophobic prodrug with the cell membrane, similar to what is described in the literature as lateral diffusion.

Although rapidly hydrolysable prodrug was used for this experiment, more slowly hydrolysable prodrugs can be used with isolated perfusion delivery methods without increasing systemic toxicity.

### Example 13

Enhanced prodrug uptake by hepatic metastases following single bolus injection. All animal experiments were performed in accordance with Institutional Animal Care and Use Committee protocols. All surgical procedures were performed under Isoflurane anesthesia. Liver tumors models were established in C57BL mice using MC38 (colon carcinoma) and Hepa 1-6 (hepatoma), in BALB/c mice using B16 (melanoma), and in A/J mice using NXS2 (neuroblastoma) cell lines. Mice were inoculated via the portal vein with 1×10⁴ MC38 cells or 0.5-1.25×10⁶ Hepa 1-6 cells, or via the tail vein with 0.5-1.25×10⁶ B16 or NXS2 cells. Tumor formation was allowed to progress for 2-3 weeks with periodic examination for tumor growth prior to drug/prodrug treatment. Microscopically, all tumors were arterialized with minimal necrosis or apoptosis.

Drug/prodrug solutions were delivered via a liver arterial bolus injection (LABI) to the right gastro-duodenal artery for retrograde delivery to the hepatic artery similar to procedures used clinically. The right gastro-duodenal artery was freed from surrounding tissue and the common hepatic artery was clamped occluding blood flow. The distal part of the right gastroduodenal artery was sutured, a 35 G needle was inserted into the gastroduodenal artery, and secured during the single bolus injection. Following the injection, the needle was retracted and the proximal part of gastroduodenal artery was sutured, and hepatic artery flow was restored. Alternatively, the celiac trunk was clamped close to the aorta and a 35 G needle was inserted above the clamp. The left gastric, splenic, and gastro-duodenal arteries were clamped in order to direct all of the drug solution to the liver. This latter approach was advantageous in the C57BL mouse model because of anatomical variations involving the hepatic artery.

LABI delivery of drug/prodrug (0.150 μmol, 220 μl total injection volume) and controls were performed using the mixing chamber and syringe pumps as previously described. For fluorescent microscopy analysis, the livers were harvested 5 min after LABI, snap frozen in O.C.T. compound, sectioned, stained (for confocal, green—actin (Alexa 488), blue—nuclei (ToPro-3)), and analyzed by fluorescent (Axioplan2) fluorescent microscope (Zeiss) in the rhodamine fluorescence channel (λₑₓ=BP 450/12, λₑₘ=LP 590 nm filter set) or in the fluorescein fluorescent channel (λₑₓ=BP 546/90, λₑₘ=LP 515 nm filter set) due to signal intensity) and confocal microscopy (Axioplan 2, Zeiss). For experiments designed to determine the number of PI positive cells, five random fields containing liver tumors and five random fields containing portal triads void of tumor were imaged. The number of cells and positive cells were counted to determine the percentage of positive cells in the corresponding regions.

For intraportal injections both the portal vein and the celiac artery were clamped to occlude blood flow into the liver. In other experiments, the portal vein and the celiac artery were not clamped, preserving full portal blood flow during the experiment. In all cases, the vena cava was not clamped in order to avoid increased pressure within the liver during the injection procedure. For experiments with occluded blood flow, the blood in the liver was flushed out with 1 ml of ITG delivered through the portal vein (1 min). Then 0.300 μmol of PI or II in 40 μl of DMF was delivered together with 400 μl of ITG via the mixing chamber. Five min after injection the livers were perfused with 3 ml of ITG to flush any drug remaining in the vasculature, and livers were sectioned as above or the hepatocytes were isolated.

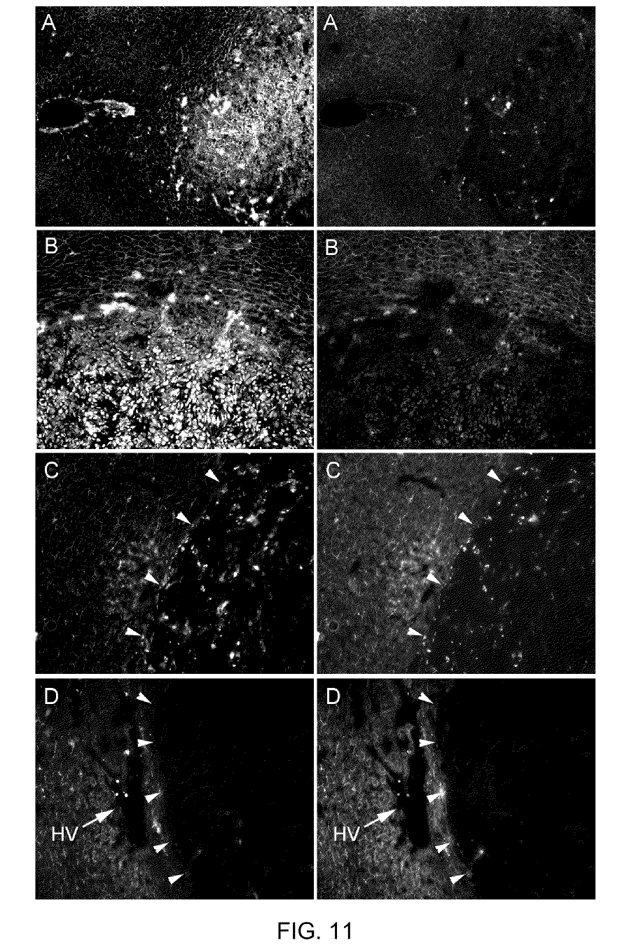

Results: In order to test in vivo tumor utility, a tumor model was established in mice with MC38 (mouse colon carcinoma) cells. All animals were examined for tumor development prior to testing with drug or RRH-prodrug, to insure that the developed tumors were large enough to insure hepatic arterialization (generally 3-5 mm, 2 to 3 weeks post inoculation). Following three weeks of tumor development PI and RRH-PI prodrug solutions were delivered using the mixing chamber via a liver arterial bolus injection (LABI). As shown in FIG. 11A-B, left panel, delivery of BDMODS-PI resulted in intense, near-exclusive PI-staining of MC38 liver metastases, while normal parenchyma appeared relatively free of PI staining. Hepatic arteries and some adjunct cells were also labeled, as were a few cells at the parenchyma-metastasis interface. No PI positive cells were observed in any other organ examined (heart, lung, spleen, and kidneys) in any of the II treated animals, indicating that II was hydrolyzed back to PI prior to encountering other tissues. LABI with III resulted in a similar cellular uptake pattern as that observed with II (data not shown). LABI with PI resulted in a greatly decreased number of PI positive cells within the MC38 metastases, which were presumed to be necrotic or apoptotic cells (FIG. 11C, left panel). Similar results were obtained when II was premixed with ITG (10 min) before LABI (data not shown). Analysis of liver sections by confocal microscopy following delivery of II again indicated widespread PI positive cells within the MC38 metastases and along the portal. Regions of highly positive PI staining in the tumors appeared to correspond to regions that were highly arterialized (FIG. 11). Confocal microscopy showed that the endothelial cells of the hepatic artery were also PI positive following LABI delivery of II. Few PI positive cells were observed after injection of PI.

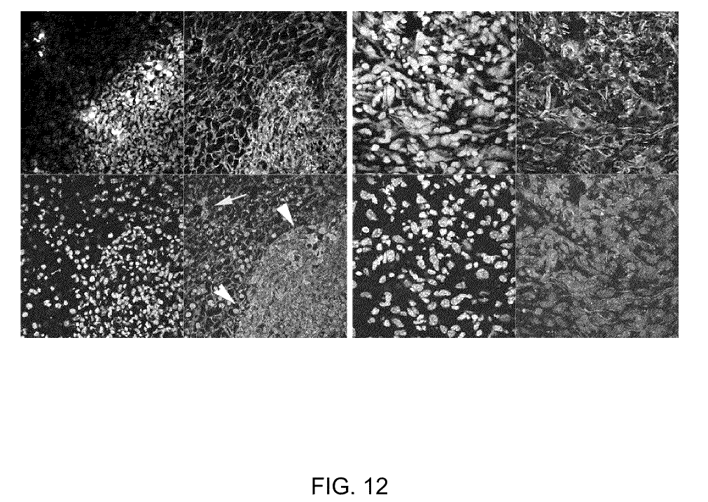

Confocal imaging was also utilized to approximate the numbers of PI positive cells following LABI in three different regions of the liver: a) MC38 metastases, b) portal vein areas of the liver, and c) liver parenchyma representing zone 2 of the hepatic unit. Five images (0.262 mm² each) were obtained for each compartment from a lobe with MC38 tumors and the total number of nuclei (ToPro-3-positive staining) and PI positive nuclei were determined. LABI with II resulted in an average of 94% of cells in MC38 metastases being PI positive. In the portal vein region, an average of 23% of the cells were PI positive. PI positive cells in this region were comprised of arterial cells, some adjoining hepatocytes, and bile duct cells (all of which are exposed to II during the injection). In the liver parenchyma region, an average of 1% of cells were PI positive. These data were used to estimate the overall percentage of non tumor cells in the liver that were PI positive. As the portal vein region comprises less than 5-7% of the liver (in humans, similar in rodent), the total number of PI positive non tumor cells in the liver can be estimated to be 2-3% (FIG. 12).

To demonstrate the arterial supply difference between tumor and hepatocytes, RRH-PI prodrug was delivered into the portal vein of normal and tumor-bearing mice with no clamping of liver outflow. When II was injected into the portal vein of tumor-bearing mice with preserved portal blood flow, all metastases were PI-negative, and few hepatic cells were positive for PI (FIG. 11D). Most likely, the RRH-PI were sequestered by blood constituents, such as erythrocytes, effectively lowering the bioavailability of the prodrug. When intraportal delivery was again performed with the portal vein and celiac artery transiently clamped to occlude blood flow (no clamping on liver outflow), there was prominent labeling of portal structures and adjacent cells, including hepatocytes, but not of the metastases. As with arterial injections, injection of unmodified PI into the portal vein (with occluded blood flow) resulted in a few labeled cells, either within the portal structure or hepatocytes.

In addition to the MC38 colon carcinoma model, we also performed preliminary testing of RRH-PI delivery in other relevant syngeneic murine liver metastases models, including Hepa 1-6 (hepatoma), B16 (melanoma), and NXS2 (neuroblastoma). Similar levels of PI positive cells were observed, with preferential staining of hepatic metastatic tissue and very minimal involvement of parenchymal cells (data not shown). Additionally, testing with III was similarly investigated for cellular uptake in vivo. In all cases, the results with III paralleled the results detailed with II (data not shown). Taken together, these results supported our idea that the RRH approach enabled selective and first-pass delivery of a drug to a tumor in vivo.

### Example 14

Uptake of BDMODS-PI by hepatocytes in vivo. Intraportal injections were conducted in order to target hepatocytes. For the injections, both the portal vein and the celiac artery were clamped to occlude blood flow into the liver. The vena cava was not clamped in order to avoid increased pressure within the liver during the injection procedure. The blood in the liver was flushed out with 1 ml of ITG delivered through the portal vein (1 min). 0.300 μmol PI or II in 40 μl of DMF was then delivered together with 400 μl ITG via the mixing chamber. 5 min after injection the livers were perfused with 3 ml of ITG to flush any drug remaining in the vasculature, and livers were sectioned as above or the hepatocytes were isolated. Slides were prepared from the resulting hepatocyte isolations for examination by fluorescent microscopy (Axioplan2 fluorescent microscope in the fluorescein fluorescent channel (λₑₓ=BP 546/90, λₑₘ=LP 515 nm filter set) due to signal intensity, magnification=400×, Zeiss) by placing a drop of the cell suspension (about 20 μl) on a clean glass slide and preparing a cell smear. Random fields were examined, and the number of positive cells was counted out of a total of 200 cells in order to determine the percentage of PI positive cells in the sample.

The amount of PI retained in hepatocytes was also quantitated following intraportal injections of PI and II. The isolated hepatocyte cell suspensions were dissolved in 0.5% octyl glycoside in 10 mM HEPES buffer, pH 7.5. Nucleic acid was then isolated from cell suspensions (0.5 ml) using phenol extraction. PI fluorescence spectra were monitored (Shimadzu RF 1501 Spectrophotometer) using an excitation wavelength of 530 nm and an emission wavelength of 617 nm. All spectra were background subtracted using the fluorescence of cell suspensions from untreated animals. PI calibration curves were generated by mixing increasing amounts of PI in each of the samples. The obtained curves were linear, indicating that PI binding to nucleic acid in the samples was not saturated, and the fluorescence was a result of nucleic acid bound PI and not free PI, thus allowing for determination of the amount of PI present in the samples. The amount of PI uptake was then calculated according to the equation: Amt(%)=100×PIₛ/PIᵢ, where PIₛ is the amount of PI in the cell suspension and PIᵢ is the amount of PI or BDMOD-PI injected (corrected for the sample size relative to the total liver).

Results: Analyses of cell suspensions prepared from normal (non tumor bearing) liver samples following portal vein injections (occluded blood flow) indicated that the injection of II resulted in approximately 70% of the cells (hepatocytes) being PI positive. The corresponding injection of PI to normal liver resulted in approximately 1% of the cells being PI positive. Nucleic acid (DNA and RNA) was also isolated from the cell suspensions by phenol extraction in order to estimate the amount of PI taken up by the hepatocytes. The nucleic acid samples were treated with increasing amounts of PI and analyzed by fluorescent spectroscopy. The result was a linear response, indicating that the isolated nucleic acid had not been saturated with PI during the intraportal injection. Therefore, and estimation of the amount of bound PI in the nucleic acid was possible. The results from this analysis indicated that 1.2% of the injected unmodified PI was bound to hepatocyte nucleic acid compared to 14% for BDMOD-PI. This estimation accounts for PI that was bound to the nucleic acid in the hepatocytes, and not to PI that was still associated with the membrane or unbound within the cell, which would have been lost during nucleic acid isolation.

### Example 15

Delivery of RRH-PI to rapidly dividing cells results in a decrease in the number of cells in mitosis. The effect of drug/prodrug treatment was also evaluated on rapidly dividing cells following a 70% partial hepatectomy on normal ICR mice. Following the hepatectomy, PI or III (0.150 μmol, 220 μl total injection volume) were delivered to the liver via the portal vein using the mixing chamber. The abdominal cavity was closed in two layers with 4-0 Braunamid suture. After 48 h, the animals were sacrificed, the livers were harvested, formalin fixed, paraffin imbedded, sectioned, and H&E stained. The sections were examined by microscopy on an Axioplan2 fluorescent microscope (Zeiss). 50 random parenchymal fields per animal were examined and the number of hepatocyte mitotic figures (metaphase) were determined together with the total number of hepatocytes (approximately 2000 per animal). Results are reported as the percentage of cells in metaphase (±standard deviation).

Results: PI and III were intraportally injected (occluded blood flow) into normal mice immediately after being subjected to a 70% hepatectomy in order to evaluate their effect on rapidly dividing cells. At 2 days after treatment with PI an average of 7.0% (±2.2) of the hepatocytes were in metaphase. In contrast, after treatment with III an average of 2.3% (±2.9) of the hepatocytes were in metaphase. These results are indicative of an antiproliferative effect for III.

### Example 16

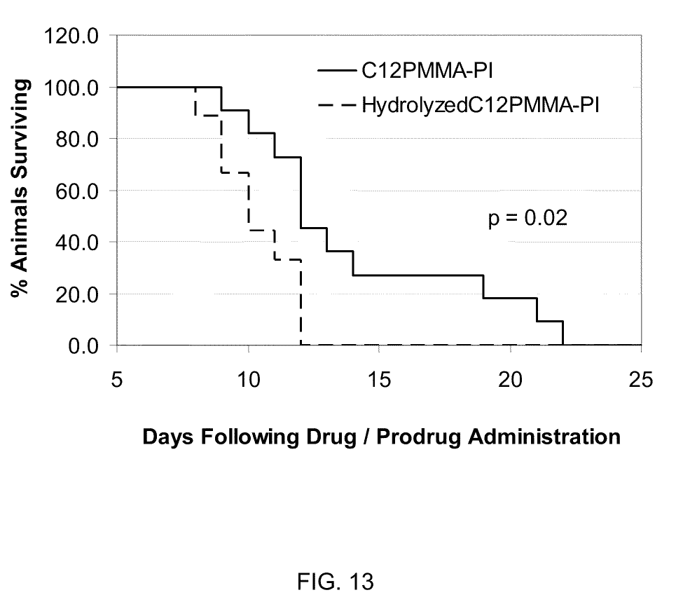

In vivo antitumor effect following a liver arterial bolus injection with a RRH-PI to MC 38 tumor bearing mice. LABI was conducted as previously described in order to monitor the antitumor effect of an RRH-PI prodrug. In the experiment, III (n=11) or hydrolyzed C12PMMA-PI (n=9) were injected as previously described, the mouse abdomen was closed after drug treatment and the animals were monitored for survival time. Statistical analysis was conducted using a t-test (two tailed distribution and two sample—unequal variance, Excel). Animals that received III exhibited increased survival (ρ=0.02), compared to the animals that received the hydrolyzed C12PMMA-PI (HyC12PMMA-PI, FIG. 13).

### Example 17

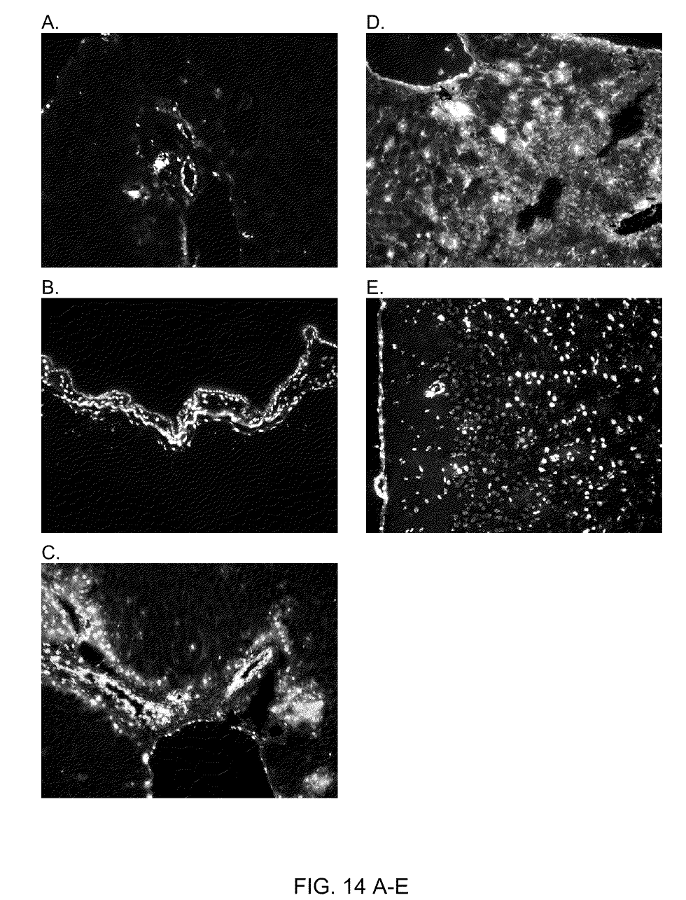

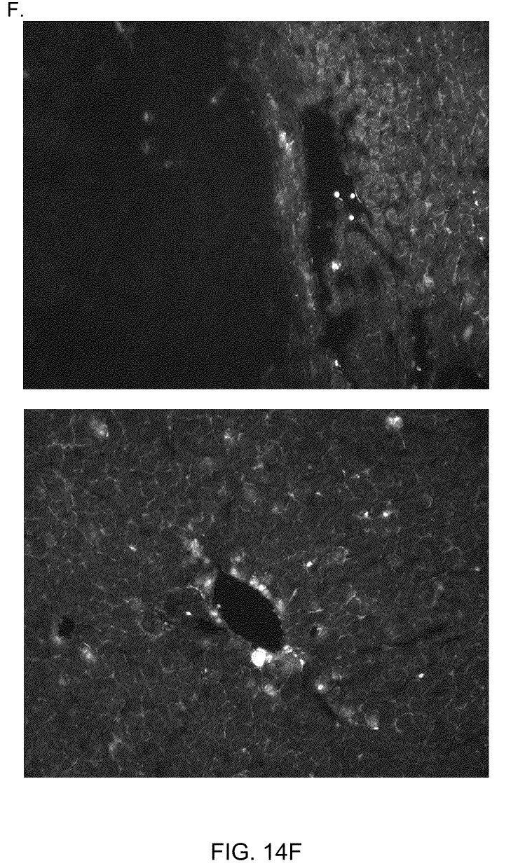

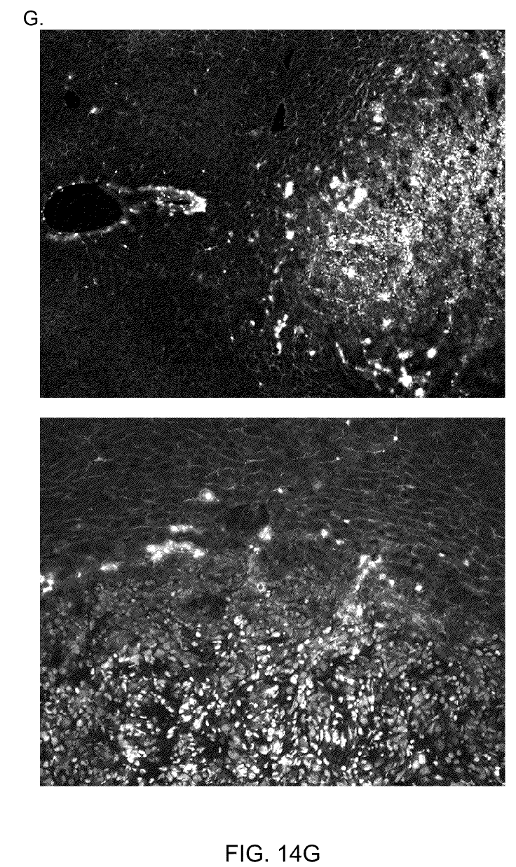

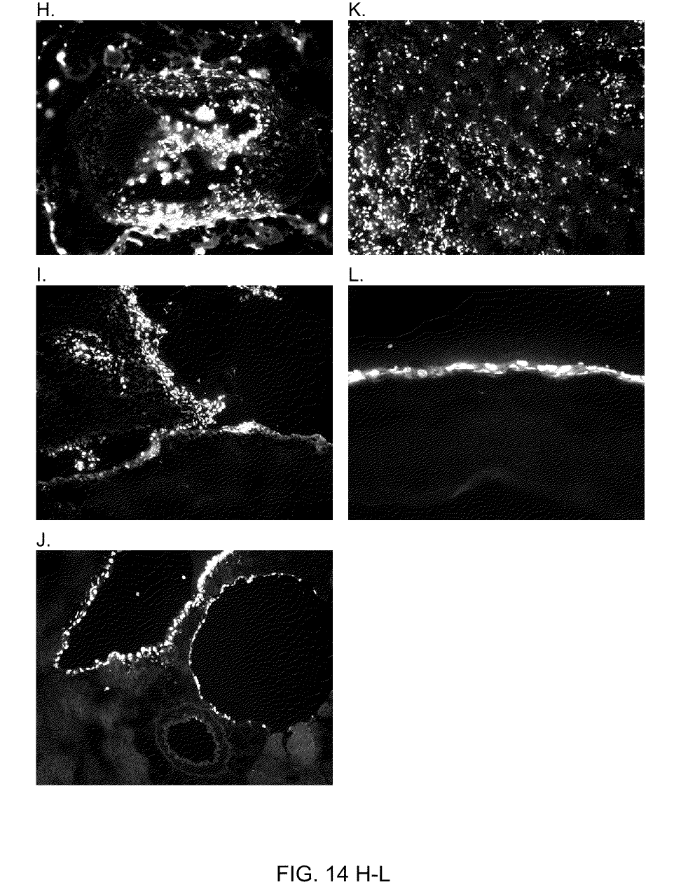

Propidium iodide delivery to a variety of target cells. Propidium iodide and RRH-PI prodrugs were delivered via injection or topical administration using the mixing chamber and as detailed below.
A. Injection into the hepatic artery of normal mouse liver: Injection of unmodified propidium iodide into any vessel or bile duct of normal liver resulted in little to no nuclear staining. Injection of II into the hepatic artery of normal mouse liver resulted in strong nuclear staining of hepatic artery endothelial and smooth-muscle cells (FIG. 14A), and stained a few neighboring hepatocytes and sinusoidal cells. All biliary and gall bladder arteries, as well as bladder epithelium also stained (FIG. 14B). The technique described above was utilized.
B. Injection into the bile duct of normal mouse liver: Injection of II into bile duct of normal mouse liver resulted in strong nuclear staining of all bile duct epithelial cells as well as staining in hepatocytes near the bile duct (FIG. 14C).
C. Injection into the portal vein of normal mouse liver: Injection of II into unclamped liver portal vein with preserved blood flow of normal mouse resulted in strong nuclear staining of portal vein cells only. When the portal vein was clamped during the injection, propidium iodide staining was observed in a majority of hepatocytes (FIG. 14D).
D. Injection into the right carotid artery of normal mouse: 35 G needle was inserted into right common carotid artery then advanced into internal carotid. During injection time the common carotid artery was temporary occluded. After injection the blood flow was restored and animals sacrificed 5 min later. Injection of II into right carotid artery of normal mouse resulted in strong staining of brain endothelial cells and in strong staining of both neuron and glial nuclei (FIG. 14E).
E. Injection into the hepatic artery of mouse liver with cancer metastases: Mouse livers were inoculated with MC38 colon carcinoma cells. After three weeks, mice were injected with modified PI. Injection of 400 μl propidium iodide into the portal vein of mouse liver with cancer metastases did not result in nuclear staining of any structures (FIG. 14F, 200×, top panel—metastisis, bottom panel—portal triad). However, injection of 350 μl II into hepatic artery of mouse liver with cancer metastases resulted in strong nuclear staining of hepatic artery endothelial and smooth-muscle cells, and in strong nuclear staining of the metastases (FIG. 14G, top panel—100×, bottom panel—200×).
F. Injection into the ureter and bladder of normal mice: Injection of II into the ureter of normal mice resulted in strong staining of ureter transitional epithelium nuclei (FIG. 14H), renal pelvis transitional epithelium nuclei (FIG. 14I), including beginning renal pelvis (FIG. 14J), and a majority of collecting tubules (FIG. 14K). Injection was performed using similar 35 G needle into right ureter close to the bladder. Injection into emptied bladder resulted strong staining of bladder transitional epithelium.
G. Application on the right cornea of normal mice: Using the described above technique, the topical application to the cornea of normal mice resulted in strong nuclear staining of cornea epithelium only (FIG. 14L).
H. Application on skin surface of normal mice: Using the described above technique, the topical application to a skin of normal mice resulted in strong nuclear staining of epidermis predominantly (data not shown).
I. Intestinal intra lumen application of the BDMODS-PI: About 25 mm of duodenum and jejunum were clamped proximally and distally then 100 μg of PI or BDMODS-PI in 20 μl of DMF were mixed with 200 μl ITG and injected into lumen over 30 s, using the technique described above. Clamps were then released and 5 min later animals were sacrificed. The intestine lumen was flushed with 1 ml of ITG and analyzed as described above. While PI staining was observed in a few intestinal villi cells (presumably apoptotic cells), application of BDMODS-PI resulted in strong labeling of all enterocyte nuclei and significant portion of mesenchymal cells near enterocytes (data not shown).

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. A method for treating a tumor in a mammal comprising:
a) covalently linking a hydrophobic group to an antitumor drug via a rapidly reversible linkage thereby forming a rapidly reversible hydrophobized antitumor drug wherein the rapidly reversible hydrophobized antitumor drug is synthesized in or dissolved in a suitable solvent in which the rapidly reversible linkage is stable;
b) mixing the rapidly reversible hydrophobized antitumor drug in the suitable solvent with a pharmaceutically acceptable carrier solution to form a delivery solution, wherein the rapidly reversible linkage is unstable in the delivery solution; and,
c) administering said delivery solution to the mammal.

2. The method of claim 1 wherein the rapidly reversible hydrophobized antitumor drug is more membrane permeable than the antitumor drug.

3. The method of claim 2 wherein said hydrophobic group is selected from the list consisting of: an alkyl chain having 4 to 30 carbon atoms, an alkyl group containing an alkyl chain and alkyl rings, and steroid.

4. The method of claim 2 wherein said suitable solvent consists of as organic solvent.

5. The method of claim 2 wherein said labile linkage consists of a hydrolytically labile bond.

6. The method of claim 5 wherein said hydrolytically labile linkage is selected from the list consisting of: silazane and maleamic acid.

7. The method of claim 5 wherein said carrier solution consists of an aqueous solution.

8. The method of claim 1 wherein said labile linkage consists of a linkage that is cleaved by a component of said carrier solution.

9. The method of claim 1 wherein said antitumor drug is selected from the group consisting of: chemotherapeutic drug, anti-neoplastic drug, active derivative of the drug containing a functional group suitable for modification, doxorubicin, cisplatin, melphalan, and paclitaxel.

10. The method of claim 1 wherein the tumor consists of a solid tumor.

11. The method of claim 10 wherein administering said delivery solution to the mammal comprises administering the delivery solution at or near the tumor cell.

12. The method of claim 11 wherein administering said delivery solution to the mammal comprises directly applying the delivery solution to the solid tumor.

13. The method of claim 10 wherein the solid tumor consists of a vascularized tumor.

14. The method of claim 13 wherein administering said delivery solution to the mammal comprises inserting the delivery solution into a vessel.

15. The method of claim 14 wherein inserting the delivery solution into a vessel comprises a single bolus injection into a vessel of the tumor or a tissue containing the tumor.

15. The method of claim 14 wherein inserting the delivery solution into a vessel comprises perfusion of the tumor or a tissue containing the tumor.

16. The method of claim 2 wherein the rapidly reversible linkage has a half-life less that 2 minute in the delivery solution.

17. The method of claim 16 wherein the rapidly reversible linkage has a half-life less that 1 minute in the delivery solution.

18. The method of claim 17 wherein the rapidly reversible linkage has a half-life less that 30 seconds in the delivery solution.

19. The method of claim 18 wherein the rapidly reversible linkage has a half-life less that 20 seconds in the delivery solution.

20. The method of claim 1 wherein the tumor is selected from the group consisting of: single cells, microinfiltrates, microtumors, larger tumors, tumor suspended in a peritoneal cavity, tumor attached to an organ or tissue, and tumor invading an organ or tissue.

21. The method of claim 1 wherein the tumor is selected from the group consisting of: cancer cell, metastatic cancer cell, liver cancer, metastatic liver cancer, hepatoma, carcinoma, hepatocellular carcinoma, colon carcinoma, ovarian carcinoma, peritoneal cancer, disseminated peritoneal ovarian cancer, melanoma, and neuroblastoma.
